(19) United States
(12) Patent Application Publication
(10) Pub. No.: US 2022/0028538 A1
(43) Pub. Date: Jan. 27, 2022

(54) METRIC-BASED DIGITAL FEED PERFORMANCE MODEL

(71) Applicant: Alegeus Technologies, LLC, Waltham, MA (US)

(72) Inventors: Steven Auerbach, Boston, MA (US); Brian Colburn, Sudbury, MA (US); Christopher Rodkey, Beverly, MA (US)

(73) Assignee: Alegeus Technologies, LLC, Waltham, MA (US)

(21) Appl. No.: 17/214,234

(22) Filed: Mar. 26, 2021

Related U.S. Application Data
(60) Provisional application No. 63/056,320, filed on Jul. 24, 2020.

(51) Int. Cl. G16H 40/63

(52) U.S. Cl. CPC G16H 40/63

(57) ABSTRACT

Example implementations, include a system for generating a metric-based digital feed performance model, with a data processing system comprising memory and one or more processors to obtain an opportunity object for a participant service identifier generated by an opportunity engine of the data processing system based on a participant object, transmit, to a client computing device, the opportunity object responsive to the opportunity object satisfying an opportunity condition, receive, via an interface of the client computing device, an indication of a selection of the opportunity object, identify, responsive to the indication of the selection, an opportunity execution heuristic, execute, responsive to a determination that the opportunity execution heuristic satisfies an opportunity condition heuristic, the opportunity object, and modify, responsive to the execution of the opportunity object, a performance metric associated with the participant object configured to trigger an alert to improve performance.

### CROSS-REFERENCE TO RELATED PATENT APPLICATIONS

This application claims the benefit of priority under 35 U.S.C. § 119 to U.S. Provisional Patent Application No. 63/056,320, entitled “METRIC-BASED DIGITAL FEED PERFORMANCE MODEL,” filed Jul. 24, 2020, the contents of which is hereby incorporated by reference in its entirety and for all purposes as if completely and fully set forth herein.

### TECHNICAL FIELD

The present implementations relate generally to digital healthcare infrastructure, and more particularly to a metric-based digital feed performance model.

### BACKGROUND

Participants of a health care program can conduct an electronic transaction for goods or services. Due to the large number of available sources for goods or services, and the varying parameters associated with electronic transactions, it can be challenging to efficiently and accurately quantify value of a source for the goods or services rapidly enough to execute without wasting resource utilization or introducing latency or delays that may make goods or services impossible to execute timely.

### SUMMARY

Health care opportunities are increasing in complexity and scope in response to the expansion of health care services available to health care consumers. Participants in various health care support programs and health care accounts face increasingly complex and interwoven opportunities with time-sensitive conditions and complex interdependencies. However, conventional systems may not effectively quantify opportunities associated with such services and accounts effectively or timely to a participant, resulting in a loss of opportunities due to lack of computational technological systems to rapidly and securely communicate value of complex decision making opportunities with respect to a combination of individualized healthcare, financial, and geographic factors. Thus, a technological solution for a metric-based digital feed performance model is desired.

Example implementations include a system for generating a metric-based digital feed performance model, with a data processing system with a memory and one or more processors to obtain an opportunity object for a participant service identifier generated by an opportunity engine of the data processing system based on a participant object, transmit, to a client computing device, the opportunity object responsive to the opportunity object satisfying an opportunity condition, receive, via an interface of the client computing device, an indication of a selection of the opportunity object, identify, responsive to the indication of the selection, an opportunity execution heuristic, execute, responsive to a determination that the opportunity execution heuristic satisfies an opportunity condition heuristic, the opportunity object, and modify, responsive to the execution of the opportunity object, a performance metric associated with the participant object configured to trigger an alert to improve performance.

Example implementations further include an opportunity condition heuristic with a timestamp threshold, and an opportunity execution heuristic with an execution timestamp associated with the opportunity object.

Example implementations further include a data processing system to determine that the execution timestamp is before or at the timestamp threshold.

Example implementations further include an opportunity condition heuristic with a distance threshold, and an opportunity execution heuristic with at least one of an opportunity distance between an participant location associated with the participant object and an opportunity location associated with the opportunity object.

Example implementations further include a data processing system to determine that the opportunity distance is less than or equal to the distance threshold.

Example implementations further include an opportunity location with a location associated with a health service.

Example implementations further include an opportunity condition heuristic with an aggregation of a timestamp threshold and a distance threshold.

Example implementations further include a data processing system to modify, responsive to a determination that the opportunity execution heuristic does not satisfy the opportunity condition heuristic, an opportunity metric to block execution of the opportunity object.

Example implementations further include a data processing system to modify the opportunity metric based on a value of the performance metric.

Example implementations further include a data processing system to modify, responsive to a determination that the participant object satisfies a device condition, the performance metric according to a device metric.

Example implementations further include a data processing system to modify, responsive to a determination that the participant object satisfies a participant account condition, the performance metric according to a participant account metric associated with a participant account.

Example implementations also include a method for generating a metric-based digital feed performance model, by obtaining an opportunity object for a participant service identifier generated by an opportunity engine of the data processing system based on a participant object, transmitting, to a client computing device, the opportunity object responsive to the opportunity object satisfying an opportunity condition, receiving, via an interface of the client computing device, an indication of a selection of the opportunity object, identifying, responsive to the indication of the selection, an opportunity execution heuristic, executing, responsive to a determination that the opportunity execution heuristic satisfies an opportunity condition heuristic, the opportunity object, and modifying, responsive to the execution of the opportunity object, a performance metric associated with the participant object configured to trigger an alert to improve performance.

Example implementations further include an opportunity condition heuristic with a timestamp threshold, and an opportunity execution heuristic with an execution timestamp associated with the opportunity object.

Example implementations further include determining that the execution timestamp is before or at the timestamp threshold.

Example implementations further include an opportunity condition heuristic with a distance threshold, and an opportunity execution heuristic with an opportunity distance between an participant location associated with the participant object and an opportunity location associated with the opportunity object.

Example implementations further include determining that the opportunity distance is less than or equal to the distance threshold.

Example implementations further include an opportunity condition heuristic with an aggregation of a timestamp threshold and a distance threshold.

Example implementations further include modifying, responsive to a determination that the opportunity execution heuristic does not satisfy the opportunity condition heuristic, an opportunity metric to block execution of the opportunity object.

Example implementations also include a computer readable medium including one or more instructions stored thereon and executable by a processor to obtain an opportunity object for a participant service identifier generated by an opportunity engine of the data processing system based on a participant object, transmit, to a client computing device, the opportunity object responsive to the opportunity object satisfying an opportunity condition, receive, via an interface of the client computing device, an indication of a selection of the opportunity object, identify, responsive to the indication of the selection, an opportunity execution heuristic, execute, responsive to a determination that the opportunity execution heuristic satisfies an opportunity condition heuristic, the opportunity object, and modify, responsive to the execution of the opportunity object, a performance metric associated with the participant object configured to trigger an alert to improve performance.

Example implementations further include a computer readable medium with one or more instructions executable by a processor to modify, responsive to a determination that the opportunity execution heuristic does not satisfy the opportunity condition heuristic, an opportunity metric to block execution of the opportunity object.

### BRIEF DESCRIPTION OF THE DRAWINGS

These and other aspects and features of the present implementations will become apparent to those ordinarily skilled in the art upon review of the following description of specific implementations in conjunction with the accompanying figures, wherein:

FIG. 1A illustrates an example network environment including a client device in communication with a server device, in accordance with present implementations.

FIG. 1B illustrates an example cloud computing environment including a client device in communication with cloud service providers, in accordance with present implementations.

FIGS. 1C and 1D illustrate example computing devices in accordance with present implementations.

FIG. 2 illustrates an example cloud computing environment including an example data processing system, in accordance with present implementations.

FIG. 3 illustrates an example data processing system, in accordance with present implementations.

FIG. 4 illustrates an example scoring engine further to the example data processing system of FIG. 3, in accordance with present implementations.

FIG. 5 illustrates an example opportunity database system further to the example data processing system of FIG. 3, in accordance with present implementations.

FIG. 6 illustrates an example electronic device associated with an example data processing system, in accordance with present implementations.

FIG. 7 illustrates an example method of generating a metric-based digital feed performance model, in accordance with present implementations.

FIG. 8 illustrates an example method of generating a metric-based digital feed performance model further to the method of FIG. 7, in accordance with present implementations.

FIG. 9 illustrates an example method of generating a metric-based digital feed performance model further to the method of FIG. 8, in accordance with present implementations.

### DETAILED DESCRIPTION

The present implementations will now be described in detail with reference to the drawings, which are provided as illustrative examples of the implementations so as to enable those skilled in the art to practice the implementations and alternatives apparent to those skilled in the art. Notably, the figures and examples below are not meant to limit the scope of the present implementations to a single implementation, but other implementations are possible by way of interchange of some or all of the described or illustrated elements. Moreover, where certain elements of the present implementations can be partially or fully implemented using known components, only those portions of such known components that are necessary for an understanding of the present implementations will be described, and detailed descriptions of other portions of such known components will be omitted so as not to obscure the present implementations. Implementations described as being implemented in software should not be limited thereto, but can include implementations implemented in hardware, or combinations of software and hardware, and vice-versa, as will be apparent to those skilled in the art, unless otherwise specified herein. In the present specification, an implementation showing a singular component should not be considered limiting; rather, the present disclosure is intended to encompass other implementations including a plurality of the same component, and vice-versa, unless explicitly stated otherwise herein. Moreover, applicants do not intend for any term in the specification or claims to be ascribed an uncommon or special meaning unless explicitly set forth as such. Further, the present implementations encompass present and future known equivalents to the known components referred to herein by way of illustration.

A data processing system can generate quantitative values of various healthcare opportunities available to a particular user, and facilitate real-time or near-real-time execution of opportunities tailored to a particular participant of a health care service. The complexity of healthcare services and products prohibits participants of those service and products from effectively evaluating and executing opportunities for consumption of those services and products. To achieve technological solutions including rapid identification, presentation, and execution of opportunities relevant to individual health care service participants, a data processing system can communicate in real-time or near-real-time with one or more external healthcare systems linked to a participant user, and quantify opportunities linked to external insurance accounts, financial accounts, and rewards accounts, for example, for a particular participant user.

By quantification of opportunities, a data processing system can identify tangible and discrete health service and product opportunities. This quantification of opportunities linked to individual participant users can further form an analysis and execution framework and infrastructure built on a consumer-driven healthcare (“CDH”) score including for example, gamification to enable execution of financial transactions or service modifications, for example, individualized and optimized to an individual participant user. Gamification aspects of generating and leveraging a customized CDH score can include goal setting for execution of opportunities As one example, a score starts at 0 and user can work up to 100 based on taking opportunities. As another example, a score can go down based on missing opportunities or taking a 2^{nd} best opportunity. The CDH score can also reflect a quantified value of a transaction based on an absolute or relative time, or on an expiration or activation threshold, for example. The data processing system can monitor user transactions to determine whether an opportunity has been taken or missed within a predetermined time interval of when the opportunity has been presented. With respect to transaction processing and service modification, for example, the data processing system can take into account factors to determine the CDH score including, but not limited to, present value of dollar savings, future value of dollar savings, amount of effort associated with an opportunity. Specifically, an amount of effort can be based on factors including time and distance to travel to obtain a product or service, and can vary by, for example, a differentiation between a best opportunity and a second-best opportunity. As one example, a second-best opportunity can reflect a lower cost savings, or a longer distance, for example. The data processing system can also modify or weight CDH score based on status, memberships, or enrollments, for example, of participant user. As one example, a CDH score can be modified based on whether a participant is enrolled in a health savings account (“HSA”), and can reflect an “HSA score” or “non-HSA score” accordingly. The CDH score can thus further the technological solution of rapidly identifying, analyzing, and presenting individualized healthcare opportunities to a participant user. For purposes of reading the description of the various implementations below, the following descriptions of the sections of the specification and their respective contents can be helpful:

Section A describes a network environment and computing environment which can be useful for practicing implementations described herein.

Section B describes implementations of systems and methods for a metric-based digital feed performance model.

### A. Computing and Network Environment

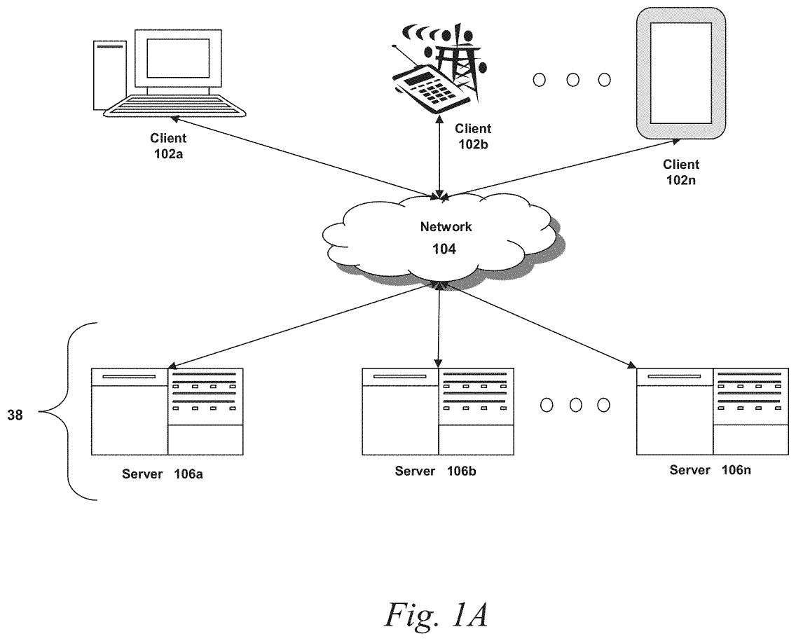

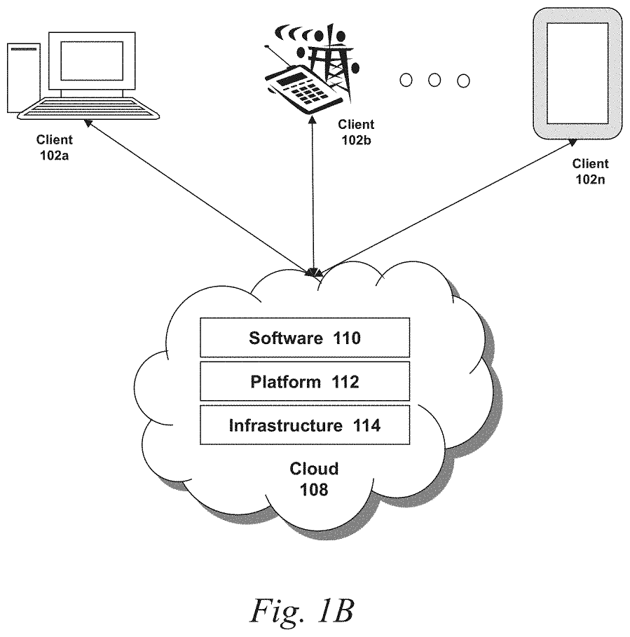

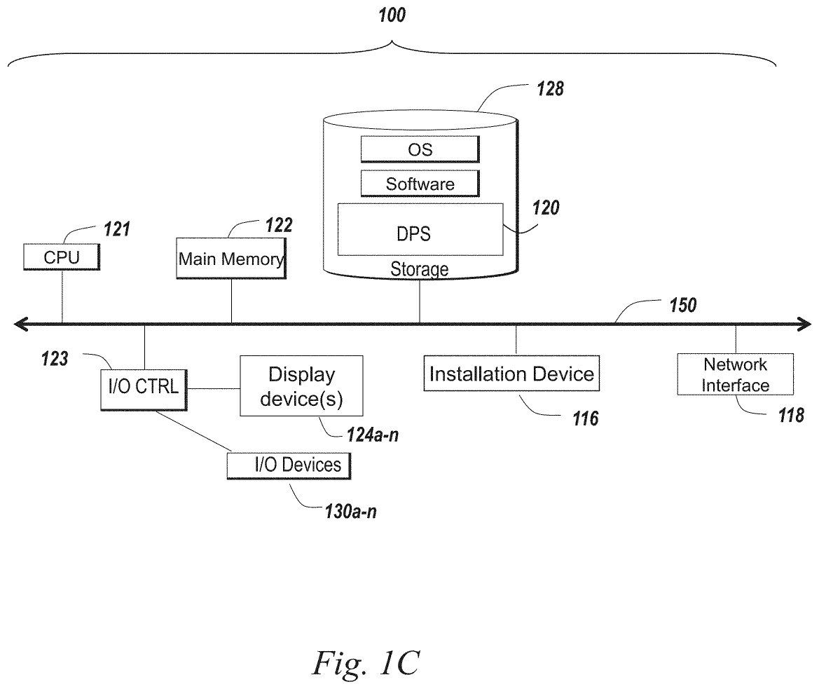

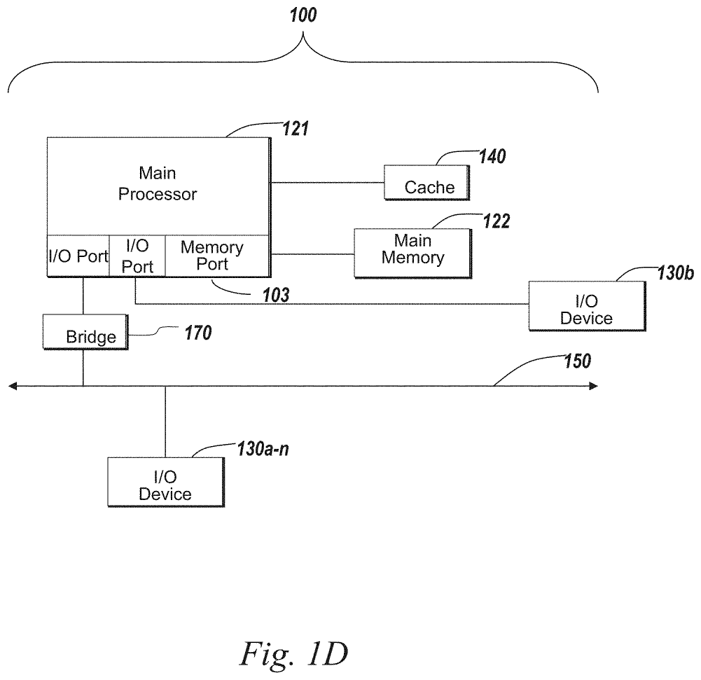

Prior to discussing specific implementations of the present solution, it can be helpful to describe aspects of the operating environment as well as associated system components (e.g., hardware elements) in connection with the methods and systems described herein. Referring to FIG. 1A, an implementation of a network environment is depicted. In brief overview, the network environment includes one or more clients 102a-102n (also generally referred to as local machine(s) 102, client(s) 102, client node(s) 102, client machine(s) 102, client computer(s) 102, client device(s) 102, endpoint(s) 102, or endpoint node(s) 102) in communication with one or more servers 106a-106n (also generally referred to as server(s) 106, node 106, or remote machine(s) 106) via one or more networks 104. In some implementations, a client 102 has the capacity to function as both a client node seeking access to resources provided by a server and as a server providing access to hosted resources for other clients 102a-102n.

Although FIG. 1A shows a network 104 between the clients 102 and the servers 106, the clients 102 and the servers 106 can be on the same network 104. In some implementations, there are multiple networks 104 between the clients 102 and the servers 106. In one of these implementations, a network 104′ (not shown) can be a private network and a network 104 can be a public network. In another of these implementations, a network 104 can be a private network and a network 104′ a public network. In still another of these implementations, networks 104 and 104′ can both be private networks.

The network 104 can be connected via wired or wireless links. Wired links can include Digital Subscriber Line (DSL), coaxial cable lines, or optical fiber lines. The wireless links can include BLUETOOTH, Wi-Fi, Worldwide Interoperability for Microwave Access (WiMAX), an infrared channel or satellite band. The wireless links can also include any cellular network standards used to communicate among mobile devices, including standards that qualify as 1G, 2G, 3G, or 4G. The network standards can qualify as one or more generation of mobile telecommunication standards by fulfilling a specification or standards such as the specifications maintained by International Telecommunication Union. The 3G standards, for example, can correspond to the International Mobile Telecommunications-2000 (IMT-2000) specification, and the 4G standards can correspond to the International Mobile Telecommunications Advanced (IMT-Advanced) specification. Examples of cellular network standards include AMPS, GSM, GPRS, UMTS, LTE, LTE Advanced, Mobile WiMAX, and WiMAX-Advanced. Cellular network standards can use various channel access methods e.g. FDMA, TDMA, CDMA, or SDMA. In some implementations, different types of data can be transmitted via different links and standards. In other implementations, the same types of data can be transmitted via different links and standards.

The network 104 can be any type and/or form of network. The geographical scope of the network 104 can vary widely and the network 104 can be a body area network (BAN), a personal area network (PAN), a local-area network (LAN), e.g. Intranet, a metropolitan area network (MAN), a wide area network (WAN), or the Internet. The topology of the network 104 can be of any form and can include, e.g., any of the following: point-to-point, bus, star, ring, mesh, or tree. The network 104 can be an overlay network which is virtual and sits on top of one or more layers of other networks 104′. The network 104 can be of any such network topology as known to those ordinarily skilled in the art capable of supporting the operations described herein. The network 104 can utilize different techniques and layers or stacks of protocols, including, e.g., the Ethernet protocol, the internet protocol suite (TCP/IP), the ATM (Asynchronous Transfer Mode) technique, the SONET (Synchronous Optical Networking) protocol, or the SDH (Synchronous Digital Hierarchy) protocol. The TCP/IP internet protocol suite can include application layer, transport layer, internet layer (including, e.g., IPv6), or the link layer. The network 104 can be a type of a broadcast network, a telecommunications network, a data communication network, or a computer network.

In some implementations, the system can include multiple, logically-grouped servers 106. In one of these implementations, the logical group of servers can be referred to as a server farm 38 or a machine farm 38. In another of these implementations, the servers 106 can be geographically dispersed. In other implementations, a machine farm 38 can be administered as a single entity. In still other implementations, the machine farm 38 includes a plurality of machine farms 38. The servers 106 within each machine farm 38 can be heterogeneous—one or more of the servers 106 or machines 106 can operate according to one type of operating system platform (e.g., WINDOWS NT, manufactured by Microsoft Corp. of Redmond, Wash.), while one or more of the other servers 106 can operate on according to another type of operating system platform (e.g., Unix, Linux, or Mac OS X).

In one implementation, servers 106 in the machine farm 38 can be stored in high-density rack systems, along with associated storage systems, and located in an enterprise data center. In this implementation, consolidating the servers 106 in this way can improve system manageability, data security, the physical security of the system, and system performance by locating servers 106 and high performance storage systems on localized high performance networks. Centralizing the servers 106 and storage systems and coupling them with advanced system management tools allows more efficient use of server resources.

The servers 106 of each machine farm 38 do not need to be physically proximate to another server 106 in the same machine farm 38. Thus, the group of servers 106 logically grouped as a machine farm 38 can be interconnected using a wide-area network (WAN) connection or a metropolitan-area network (MAN) connection. For example, a machine farm 38 can include servers 106 physically located in different continents or different regions of a continent, country, state, city, campus, or room. Data transmission speeds between servers 106 in the machine farm 38 can be increased if the servers 106 are connected using a local-area network (LAN) connection or some form of direct connection. Additionally, a heterogeneous machine farm 38 can include one or more servers 106 operating according to a type of operating system, while one or more other servers 106 execute one or more types of hypervisors rather than operating systems. In these implementations, hypervisors can be used to emulate virtual hardware, partition physical hardware, virtualize physical hardware, and execute virtual machines that provide access to computing environments, allowing multiple operating systems to run concurrently on a host computer. Native hypervisors can run directly on the host computer. Hypervisors can include VMware ESX/ESXi, manufactured by VMWare, Inc., of Palo Alto, Calif.; the Xen hypervisor, an open source product whose development is overseen by Citrix Systems, Inc.; the HYPER-V hypervisors provided by Microsoft or others. Hosted hypervisors can run within an operating system on a second software level. Examples of hosted hypervisors can include VMware Workstation and VIRTUALBOX.

Management of the machine farm 38 can be de-centralized. For example, one or more servers 106 can comprise components, subsystems and modules to support one or more management services for the machine farm 38. In one of these implementations, one or more servers 106 provide functionality for management of dynamic data, including techniques for handling failover, data replication, and increasing the robustness of the machine farm 38. Each server 106 can communicate with a persistent store and, in some implementations, with a dynamic store.

Server 106 can be a file server, application server, web server, proxy server, appliance, network appliance, gateway, gateway server, virtualization server, deployment server, SSL VPN server, or firewall. In one implementation, the server 106 can be referred to as a remote machine or a node. In another implementation, a plurality of nodes 290 can be in the path between any two communicating servers.

Referring to FIG. 1B, a cloud computing environment is depicted. A cloud computing environment can provide client 102 with one or more resources provided by a network environment. The cloud computing environment can include one or more clients 102a-102n, in communication with the cloud 108 over one or more networks 104. Clients 102 can include, e.g., thick clients, thin clients, and zero clients. A thick client can provide at least some functionality even when disconnected from the cloud 108 or servers 106. A thin client or a zero client can depend on the connection to the cloud 108 or server 106 to provide functionality. A zero client can depend on the cloud 108 or other networks 104 or servers 106 to retrieve operating system data for the client device. The cloud 108 can include back end platforms, e.g., servers 106, storage, server farms or data centers.

The cloud 108 can be public, private, or hybrid. Public clouds can include public servers 106 that are maintained by third parties to the clients 102 or the owners of the clients. The servers 106 can be located off-site in remote geographical locations as disclosed above or otherwise. Public clouds can be connected to the servers 106 over a public network. Private clouds can include private servers 106 that are physically maintained by clients 102 or owners of clients. Private clouds can be connected to the servers 106 over a private network 104. Hybrid clouds 108 can include both the private and public networks 104 and servers 106.

The cloud 108 can also include a cloud based delivery, e.g. Software as a Service (SaaS) 110, Platform as a Service (PaaS) 112, and Infrastructure as a Service (IaaS) 114. IaaS can refer to a user renting the use of infrastructure resources that are needed during a specified time period. IaaS providers can offer storage, networking, servers or virtualization resources from large pools, allowing the users to quickly scale up by accessing more resources as needed. Examples of IaaS can include infrastructure and services (e.g., EG-32) provided by OVH HOSTING of Montreal, Quebec, Canada, AMAZON WEB SERVICES provided by Amazon.com, Inc., of Seattle, Wash., RACKSPACE CLOUD provided by Rackspace US, Inc., of San Antonio, Tex., Google Compute Engine provided by Google Inc. of Mountain View, Calif., or RIGHTSCALE provided by RightScale, Inc., of Santa Barbara, Calif. PaaS providers can offer functionality provided by IaaS, including, e.g., storage, networking, servers or virtualization, as well as additional resources such as, e.g., the operating system, middleware, or runtime resources. Examples of PaaS include WINDOWS AZURE provided by Microsoft Corporation of Redmond, Wash., Google App Engine provided by Google Inc., and HEROKU provided by Heroku, Inc. of San Francisco, Calif. SaaS providers can offer the resources that PaaS provides, including storage, networking, servers, virtualization, operating system, middleware, or runtime resources. In some implementations, SaaS providers can offer additional resources including, e.g., data and application resources. Examples of SaaS include GOOGLE APPS provided by Google Inc., SALESFORCE provided by Salesforce.com Inc. of San Francisco, Calif., or OFFICE 365 provided by Microsoft Corporation. Examples of SaaS can also include data storage providers, e.g. DROPBOX provided by Dropbox, Inc. of San Francisco, Calif., Microsoft SKYDRIVE provided by Microsoft Corporation, Google Drive provided by Google Inc., or Apple ICLOUD provided by Apple Inc. of Cupertino, Calif.

Clients 102 can access IaaS resources with one or more IaaS standards, including, e.g., Amazon Elastic Compute Cloud (EC2), Open Cloud Computing Interface (OCCI), Cloud Infrastructure Management Interface (CIMI), or OpenStack standards. Some IaaS standards can allow clients access to resources over HTTP, and can use Representational State Transfer (REST) protocol or Simple Object Access Protocol (SOAP). Clients 102 can access PaaS resources with different PaaS interfaces. Some PaaS interfaces use HTTP packages, standard Java APIs, JavaMail API, Java Data Objects (JDO), Java Persistence API (JPA), Python APIs, web integration APIs for different programming languages including, e.g., Rack for Ruby, WSGI for Python, or PSGI for Perl, or other APIs that can be built on REST, HTTP, XML, or other protocols. Clients 102 can access SaaS resources through the use of web-based user interfaces, provided by a web browser (e.g. GOOGLE CHROME, Microsoft INTERNET EXPLORER, or Mozilla Firefox provided by Mozilla Foundation of Mountain View, Calif.). Clients 102 can also access SaaS resources through smartphone or tablet applications, including, e.g., Salesforce Sales Cloud, or Google Drive app. Clients 102 can also access SaaS resources through the client operating system, including, e.g., Windows file system for DROPBOX.

In some implementations, access to IaaS, PaaS, or SaaS resources can be authenticated. For example, a server or authentication server can authenticate a user via security certificates, HTTPS, or API keys. API keys can include various encryption standards such as, e.g., Advanced Encryption Standard (AES). Data resources can be sent over Transport Layer Security (TLS) or Secure Sockets Layer (SSL).

The client 102 and server 106 can be deployed as and/or executed on any type and form of computing device, e.g. a computer, network device or appliance capable of communicating on any type and form of network and performing the operations described herein. FIGS. 1C and 1D depict block diagrams of a computing device 100 useful for practicing an implementation of the client 102 or a server 106. As shown in FIGS. 1C and 1D, each computing device 100 includes a central processing unit 121, and a main memory unit 122. As shown in FIG. 1C, a computing device 100 can include a storage device 128, an installation device 116, a network interface 118, an I/O controller 123, display devices 124a-124n, a keyboard 126 and a pointing device 127, e.g. a mouse. The storage device 128 can include, without limitation, an operating system, software, and a software of a data processing system (DPS) 120. As shown in FIG. 1D, each computing device 100 can also include additional optional elements, e.g. a memory port 103, a bridge 170, one or more input/output devices 130a-130n (generally referred to using reference numeral 130), and a cache memory 140 in communication with the central processing unit 121.

The central processing unit 121 is any logic circuitry that responds to and processes instructions fetched from the main memory unit 122. In many implementations, the central processing unit 121 is provided by a microprocessor unit, e.g.: those manufactured by Intel Corporation of Mountain View, Calif.; those manufactured by Motorola Corporation of Schaumburg, Ill.; the ARM processor and TEGRA system on a chip (SoC) manufactured by Nvidia of Santa Clara, Calif.; the POWER7 processor, those manufactured by International Business Machines of White Plains, N.Y.; or those manufactured by Advanced Micro Devices of Sunnyvale, Calif. The computing device 100 can be based on any of these processors, or any other processor capable of operating as described herein. The central processing unit 121 can utilize instruction level parallelism, thread level parallelism, different levels of cache, and multi-core processors. A multi-core processor can include two or more processing units on a single computing component. Examples of multi-core processors include the AMD PHENOM IIX2, INTEL CORE i5 and INTEL CORE i7.

Main memory unit 122 can include one or more memory chips capable of storing data and allowing any storage location to be directly accessed by the microprocessor 121. Main memory unit 122 can be volatile and faster than storage 128 memory. Main memory units 122 can be Dynamic random access memory (DRAM) or any variants, including static random access memory (SRAM), Burst SRAM or SynchBurst SRAM (B SRAM), Fast Page Mode DRAM (FPM DRAM), Enhanced DRAM (EDRAM), Extended Data Output RAM (EDO RAM), Extended Data Output DRAM (EDO DRAM), Burst Extended Data Output DRAM (BEDO DRAM), Single Data Rate Synchronous DRAM (SDR SDRAM), Double Data Rate SDRAM (DDR SDRAM), Direct Rambus DRAM (DRDRAM), or Extreme Data Rate DRAM (XDR DRAM). In some implementations, the main memory 122 or the storage 128 can be non-volatile; e.g., non-volatile read access memory (NVRAM), flash memory non-volatile static RAM (nvSRAM), Ferroelectric RAM (FeRAM), Magnetoresistive RAM (MRAM), Phase-change memory (PRAM), conductive-bridging RAM (CBRAM), Silicon-Oxide-Nitride-Oxide-Silicon (SONOS), Resistive RAM (RRAM), Racetrack, Nano-RAM (NRAM), or Millipede memory. The main memory 122 can be based on any of the above described memory chips, or any other available memory chips capable of operating as described herein. In the implementation shown in FIG. 1C, the processor 121 communicates with main memory 122 via a system bus 150 (described in more detail below). FIG. 1D depicts an implementation of a computing device 100 in which the processor communicates directly with main memory 122 via a memory port 103. For example, in FIG. 1D the main memory 122 can be DRDRAM.

FIG. 1D depicts an implementation in which the main processor 121 communicates directly with cache memory 140 via a secondary bus, sometimes referred to as a backside bus. In other implementations, the main processor 121 communicates with cache memory 140 using the system bus 150. Cache memory 140 typically has a faster response time than main memory 122 and is typically provided by SRAM, BSRAM, or EDRAM. In the implementation shown in FIG. 1D, the processor 121 communicates with various I/O devices 130 via a local system bus 150. Various buses can be used to connect the central processing unit 121 to any of the I/O devices 130, including a PCI bus, a PCI-X bus, or a PCI-Express bus, or a NuBus. For implementations in which the I/O device is a video display 124, the processor 121 can use an Advanced Graphics Port (AGP) to communicate with the display 124 or the I/O controller 123 for the display 124. FIG. 1D depicts an implementation of a computer 100 in which the main processor 121 communicates directly with I/O device 130b or other processors 121′ via HYPERTRANSPORT, RAPIDIO, or INFINIBAND communications technology. FIG. 1D also depicts an implementation in which local busses and direct communication are mixed: the processor 121 communicates with I/O device 130a using a local interconnect bus while communicating with I/O device 130b directly.

A wide variety of I/O devices 130a-130n can be present in the computing device 100. Input devices can include keyboards, mice, trackpads, trackballs, touchpads, touch mice, multi-touch touchpads and touch mice, microphones, multi-array microphones, drawing tablets, cameras, single-lens reflex camera (SLR), digital SLR (DSLR), CMOS sensors, accelerometers, infrared optical sensors, pressure sensors, magnetometer sensors, angular rate sensors, depth sensors, proximity sensors, ambient light sensors, gyroscopic sensors, or other sensors. Output devices can include video displays, graphical displays, speakers, headphones, inkjet printers, laser printers, and 3D printers.

Devices 130a-130n can include a combination of multiple input or output devices, including, e.g., Microsoft KINECT, Nintendo Wiimote for the WII, Nintendo WII U GAMEPAD, or Apple IPHONE. Some devices 130a-130n allow gesture recognition inputs through combining some of the inputs and outputs. Some devices 130a-130n provides for facial recognition which can be utilized as an input for different purposes including authentication and other commands. Some devices 130a-130n provides for voice recognition and inputs, including, e.g., Microsoft KINECT, SIRI for IPHONE by Apple, Google Now or Google Voice Search.

Additional devices 130a-130n have both input and output capabilities, including, e.g., haptic feedback devices, touchscreen displays, or multi-touch displays. Touchscreen, multi-touch displays, touchpads, touch mice, or other touch sensing devices can use different technologies to sense touch, including, e.g., capacitive, surface capacitive, projected capacitive touch (PCT), in-cell capacitive, resistive, infrared, waveguide, dispersive signal touch (DST), in-cell optical, surface acoustic wave (SAW), bending wave touch (BWT), or force-based sensing technologies. Some multi-touch devices can allow two or more contact points with the surface, allowing advanced functionality including, e.g., pinch, spread, rotate, scroll, or other gestures. Some touchscreen devices, including, e.g., Microsoft PIXELSENSE or Multi-Touch Collaboration Wall, can have larger surfaces, such as on a table-top or on a wall, and can also interact with other electronic devices. Some I/O devices 130a-130n, display devices 124a-124n or group of devices can be augment reality devices. The I/O devices can be controlled by an I/O controller 123 as shown in FIG. 1C. The I/O controller can control one or more I/O devices, such as, e.g., a keyboard 126 and a pointing device 127, e.g., a mouse or optical pen. Furthermore, an I/O device can also provide storage and/or an installation medium 116 for the computing device 100. In still other implementations, the computing device 100 can provide USB connections (not shown) to receive handheld USB storage devices. In further implementations, an I/O device 130 can be a bridge between the system bus 150 and an external communication bus, e.g. a USB bus, a SCSI bus, a FireWire bus, an Ethernet bus, a Gigabit Ethernet bus, a Fibre Channel bus, or a Thunderbolt bus.

In some implementations, display devices 124a-124n can be connected to I/O controller 123. Display devices can include, e.g., liquid crystal displays (LCD), thin film transistor LCD (TFT-LCD), blue phase LCD, electronic papers (e-ink) displays, flexile displays, light emitting diode displays (LED), digital light processing (DLP) displays, liquid crystal on silicon (LCOS) displays, organic light-emitting diode (OLED) displays, active-matrix organic light-emitting diode (AMOLED) displays, liquid crystal laser displays, time-multiplexed optical shutter (TMOS) displays, or 3D displays. Examples of 3D displays can use, e.g. stereoscopy, polarization filters, active shutters, or autostereoscopy. Display devices 124a-124n can also be a head-mounted display (HMD). In some implementations, display devices 124a-124n or the corresponding I/O controllers 123 can be controlled through or have hardware support for OPENGL or DIRECTX API or other graphics libraries.

In some implementations, the computing device 100 can include or connect to multiple display devices 124a-124n, which each can be of the same or different type and/or form. As such, any of the I/O devices 130a-130n and/or the I/O controller 123 can include any type and/or form of suitable hardware, software, or combination of hardware and software to support, enable or provide for the connection and use of multiple display devices 124a-124n by the computing device 100. For example, the computing device 100 can include any type and/or form of video adapter, video card, driver, and/or library to interface, communicate, connect or otherwise use the display devices 124a-124n. In one implementation, a video adapter can include multiple connectors to interface to multiple display devices 124a-124n. In other implementations, the computing device 100 can include multiple video adapters, with each video adapter connected to one or more of the display devices 124a-124n. In some implementations, any portion of the operating system of the computing device 100 can be configured for using multiple displays 124a-124n. In other implementations, one or more of the display devices 124a-124n can be provided by one or more other computing devices 100a or 100b connected to the computing device 100, via the network 104. In some implementations software can be designed and constructed to use another computer's display device as a second display device 124a for the computing device 100. For example, in one implementation, an Apple iPad can connect to a computing device 100 and use the display of the device 100 as an additional display screen that can be used as an extended desktop. One ordinarily skilled in the art will recognize and appreciate the various ways and implementations that a computing device 100 can be configured to have multiple display devices 124a-124n.

Referring again to FIG. 1C, the computing device 100 can comprise a storage device 128 (e.g. one or more hard disk drives or redundant arrays of independent disks) for storing an operating system or other related software, and for storing application software programs such as any program related to the software 120 for the centralized state processing system. Examples of storage device 128 include, e.g., hard disk drive (HDD); optical drive including CD drive, DVD drive, or BLU-RAY drive; solid-state drive (SSD); USB flash drive; or any other device suitable for storing data. Some storage devices can include multiple volatile and non-volatile memories, including, e.g., solid state hybrid drives that combine hard disks with solid state cache. Some storage device 128 can be non-volatile, mutable, or read-only. Some storage device 128 can be internal and connect to the computing device 100 via a bus 150. Some storage device 128 can be external and connect to the computing device 100 via a I/O device 130 that provides an external bus. Some storage device 128 can connect to the computing device 100 via the network interface 118 over a network 104, including, e.g., the Remote Disk for MACBOOK AIR by Apple. Some client devices 100 may not require a non-volatile storage device 128 and can be thin clients or zero clients 102. Some storage device 128 can also be used as an installation device 116, and can be suitable for installing software and programs. Additionally, the operating system and the software can be run from a bootable medium, for example, a bootable CD, e.g. KNOPPIX, a bootable CD for GNU/Linux that is available as a GNU/Linux distribution from knoppix.net.

Client device 100 can also install software or application from an application distribution platform. Examples of application distribution platforms include the App Store for iOS provided by Apple, Inc., the Mac App Store provided by Apple, Inc., GOOGLE PLAY for Android OS provided by Google Inc., Chrome Webstore for CHROME OS provided by Google Inc., and Amazon Appstore for Android OS and KINDLE FIRE provided by Amazon.com, Inc. An application distribution platform can facilitate installation of software on a client device 102. An application distribution platform can include a repository of applications on a server 106 or a cloud 108, which the clients 102a-102n can access over a network 104. An application distribution platform can include application developed and provided by various developers. A user of a client device 102 can select, purchase and/or download an application via the application distribution platform.

Furthermore, the computing device 100 can include a network interface 118 to interface to the network 104 through a variety of connections including, but not limited to, standard telephone lines LAN or WAN links (e.g., 802.11, T1, T3, Gigabit Ethernet, Infiniband), broadband connections (e.g., ISDN, Frame Relay, ATM, Gigabit Ethernet, Ethernet-over-SONET, ADSL, VDSL, BPON, GPON, fiber optical including FiOS), wireless connections, or some combination of any or all of the above. Connections can be established using a variety of communication protocols (e.g., TCP/IP, Ethernet, ARCNET, SONET, SDH, Fiber Distributed Data Interface (FDDI), IEEE 802.11a/b/g/n/ac CDMA, GSM, WiMax and direct asynchronous connections). In one implementation, the computing device 100 communicates with other computing devices 100′ via any type and/or form of gateway or tunneling protocol e.g. Secure Socket Layer (SSL) or Transport Layer Security (TLS), or the Citrix Gateway Protocol manufactured by Citrix Systems, Inc. of Ft. Lauderdale, Fla. The network interface 118 can comprise a built-in network adapter, network interface card, PCMCIA network card, EXPRESSCARD network card, card bus network adapter, wireless network adapter, USB network adapter, modem or any other device suitable for interfacing the computing device 100 to any type of network capable of communication and performing the operations described herein.

A computing device 100 of the sort depicted in FIGS. 1B and 1C can operate under the control of an operating system, which controls scheduling of tasks and access to system resources. The computing device 100 can be running any operating system such as any of the versions of the MICROSOFT WINDOWS operating systems, the different releases of the Unix and Linux operating systems, any version of the MAC OS for Macintosh computers, any embedded operating system, any real-time operating system, any open source operating system, any proprietary operating system, any operating systems for mobile computing devices, or any other operating system capable of running on the computing device and performing the operations described herein. Typical operating systems include, but are not limited to: WINDOWS 2000, WINDOWS Server 2012, WINDOWS CE, WINDOWS Phone, WINDOWS XP, WINDOWS VISTA, and WINDOWS 7, WINDOWS RT, and WINDOWS 8 all of which are manufactured by Microsoft Corporation of Redmond, Wash.; MAC OS and iOS, manufactured by Apple, Inc. of Cupertino, Calif.; and Linux, a freely-available operating system, e.g. Linux Mint distribution (“distro”) or Ubuntu, distributed by Canonical Ltd. of London, United Kingdom; or Unix or other Unix-like derivative operating systems; and Android, designed by Google, of Mountain View, Calif., among others. Some operating systems, including, e.g., the CHROME OS by Google, can be used on zero clients or thin clients, including, e.g., CHROMEBOOKS.

The computer system 100 can be any workstation, telephone, desktop computer, laptop or notebook computer, netbook, ULTRABOOK, tablet, server, handheld computer, mobile telephone, smartphone or other portable telecommunications device, media playing device, a gaming system, mobile computing device, or any other type and/or form of computing, telecommunications or media device that is capable of communication. The computer system 100 has sufficient processor power and memory capacity to perform the operations described herein. In some implementations, the computing device 100 can have different processors, operating systems, and input devices consistent with the device. The Samsung GALAXY smartphones, e.g., operate under the control of Android operating system developed by Google, Inc. GALAXY smartphones receive input via a touch interface.

In some implementations, the computing device 100 is a gaming system. For example, the computer system 100 can comprise a PLAYSTATION 3, or PERSONAL PLAYSTATION PORTABLE (PSP), or a PLAYSTATION VITA device manufactured by the Sony Corporation of Tokyo, Japan, a NINTENDO DS, NINTENDO 3DS, NINTENDO WII, or a NINTENDO WII U device manufactured by Nintendo Co., Ltd., of Kyoto, Japan, an XBOX 360 device manufactured by the Microsoft Corporation of Redmond, Wash.

In some implementations, the computing device 100 is a digital audio player such as the Apple IPOD, IPOD Touch, and IPOD NANO lines of devices, manufactured by Apple Computer of Cupertino, Calif. Some digital audio players can have other functionality, including, e.g., a gaming system or any functionality made available by an application from a digital application distribution platform. For example, the IPOD Touch can access the Apple App Store. In some implementations, the computing device 100 is a portable media player or digital audio player supporting file formats including, but not limited to, MP3, WAV, M4A/AAC, WMA Protected AAC, AIFF, Audible audiobook, Apple Lossless audio file formats and .mov, .m4v, and .mp4 MPEG-4 (H.264/MPEG-4 AVC) video file formats.

In some implementations, the computing device 100 is a tablet e.g. the IPAD line of devices by Apple; GALAXY TAB family of devices by Samsung; or KINDLE FIRE, by Amazon.com, Inc. of Seattle, Wash. In other implementations, the computing device 100 is an eBook reader, e.g. the KINDLE family of devices by Amazon.com, or NOOK family of devices by Barnes & Noble, Inc. of New York City, N.Y.

In some implementations, the communications device 102 includes a combination of devices, e.g. a smartphone combined with a digital audio player or portable media player. For example, one of these implementations is a smartphone, e.g. the IPHONE family of smartphones manufactured by Apple, Inc.; a Samsung GALAXY family of smartphones manufactured by Samsung, Inc.; or a Motorola DROID family of smartphones. In yet another implementation, the communications device 102 is a laptop or desktop computer equipped with a web browser and a microphone and speaker system, e.g. a telephony headset. In these implementations, the communications devices 102 are web-enabled and can receive and initiate phone calls. In some implementations, a laptop or desktop computer is also equipped with a webcam or other video capture device that enables video chat and video call.

In some implementations, the status of one or more machines 102, 106 in the network 104 are monitored, generally as part of network management. In one of these implementations, the status of a machine can include an identification of load information (e.g., the number of processes on the machine, CPU and memory utilization), of port information (e.g., the number of available communication ports and the port addresses), or of session status (e.g., the duration and type of processes, and whether a process is active or idle). In another of these implementations, this information can be identified by a plurality of metrics, and the plurality of metrics can be applied at least in part towards decisions in load distribution, network traffic management, and network failure recovery as well as any aspects of operations of the present solution described herein. Aspects of the operating environments and components described above will become apparent in the context of the systems and methods disclosed herein.

### B. Metric-Based Digital Feed Performance Model

A data processing system can generate, evaluate, and prioritize, for example, quantified scores for opportunities to consume health care services and products. The data processing system can obtain one or more opportunity objects including or linked to, for example, one or more external healthcare services or products, and can generate, evaluate, reevaluate, and optimize consumption, for example, of opportunities for which quantified scores are generated and associated. Thus, a participant user of many healthcare services can be notified of the most relevant opportunities for consumption of various healthcare services in real-time or near-real-time, and can execute one or more opportunity objects with quantified score with the external healthcare systems via the data processing system. Real-time or near-real-time communication can include, but is not limited to, reducing or eliminating latency of or delay in communication to below a perceivable level by a user. As one example, latency or delay can be introduced by incompatibilities between third-party computing systems, or databases, and can be reduced or eliminated by interconnection of those systems or databases through automated communication interfaces compatible with both. The data processing system can obtain opportunity objects and participant objects linked, for example, to individual participant users and the particular healthcare services those individual participant users are enrolled in, for example.

The data processing system can execute a number of operations based on received opportunity objects, participant objects, and various external environmental metrics including, for example, time and location. The data processing system can identify a state of an opportunity by evaluating an opportunity object for multiple metrics included by that opportunity object or linked to that opportunity object. For example, the data processing system can determine a location for redeeming an opportunity, an account available from which that opportunity can be redeemed, and a time or range of times during which the opportunity is available or not available. The data processing system can also extract similar parameters or metrics, for example, associated with a participant user or participant object of the participant user for comparison with the opportunity object. For example, the data processing system can determine a universal time and a geolocation at a participant user's mobile device.

With metrics extracted from both an opportunity object and a participant object relevant to a participant user, the data processing system can evaluate the opportunity object against the participant object according to one or more heuristics. The data processing system can evaluate and validate, for example, the opportunity object based on location, time, and compatibility with a participant's enrolled services, among others. For example, the data processing system can assign a highest quantified score to an opportunity for filling a prescription at a pharmacy accepting the participant's health insurance closest to the participant's home or office. The data processing system can communicate with one or more external healthcare systems, including, for example, a participant's health insurance provider system, a pharmacy provider system, and a health savings account system, for example, to identify all metrics and heuristics relevant to an opportunity or a quantified score for that opportunity.

In response to evaluating by these heuristics, the data processing system can thus apply one or more control operations to filter, block, present, execute, or manage, for example, opportunities objects with quantitative scores indicating opportunities providing the best financial benefit, or quality of service, for example, for the individual participant.

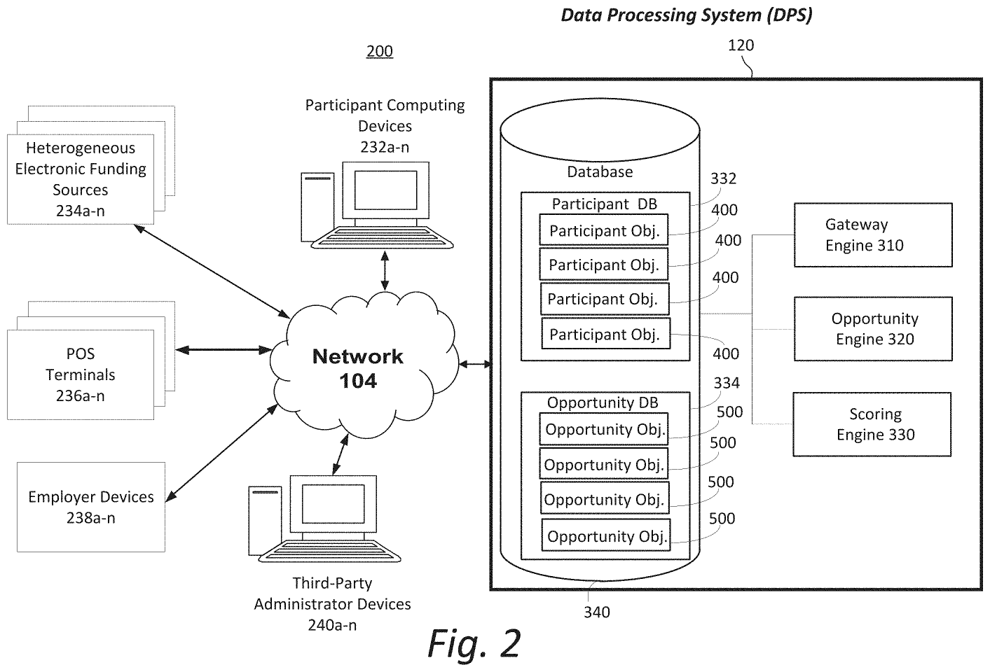

Referring now to FIG. 2, a block diagram depicting an implementation of a system 200 comprising a centralized state processing system is shown. In brief overview, the system 200 includes a data processing system 120 (“DPS”) that can receive and/or transmit data via a network 104 with participant computing devices 232a-n, third-party administrator devices 240a-n, employer devices 238a-n, point-of-sale terminals 236a-n, or heterogeneous electronic funding sources 234a-n. The DPS 120 can include a communications interface 204 that is configured with one or more communications ports, application programming interfaces, network protocols (e.g., TCP/IP), authentication protocols, or security protocols (e.g., SSL). The DPS 120 can include, interface with, or otherwise access an gateway engine 310 that controls communication, security, and the like at the DPS and components thereof. The DPS 120 can include, interface with, or otherwise access an opportunity engine 320 that analyzes, transmits, executes, and the like, opportunity objects. The DPS 120 can include, interface with, or otherwise access a scoring engine 330 that analyzes, transmits, executes, modifies, and the like, characteristics and availabilities of opportunity objects. The DPS 120 can include one or more databases or data structures that store information to facilitate the systems and methods of the present solution, such as database 340. The database 340 can include data structures, files or otherwise categorize information into different databases based at least partially by object. The database 340 can also include one or more policies, profiles, merchant information, or historical transaction activity, and the like.

The DPS 120, gateway engine 310, opportunity engine 320, and scoring engine 330 can each include one or more processing units or other logic devices such as programmable logic array engines, modules, or circuitry designed and constructed to facilitate managing security on a network infrastructure. The DPS 120 can include the components 100 shown in FIG. 1C or FIG. 1D, or be configured to operate as a service in cloud 108. The DPS 120 can include or interact with one or more servers 106a-n and clients 102a-n. The participant computing devices 232a-n, POS terminals 236a-n, employer devices 238a-n, TPA devices 240a-n, or heterogeneous electronic funding sources 234a-n can each include one or more component or functionality of client computing devices 102a-n or server 106a-n.

The DPS 120 can employ a multitier architecture such as a client-server architecture in which presentation, application processing, and data management functions are logically or physically separated. The presentation tier, or front-end, can include the communications interface 204 that can serve static content or dynamic content to be rendered by the client 102 (e.g., by a web browser executing on client 102). The presentation tier or web server can interact or communicate with the application tier to obtain data to provide to the client 102, computing devices, 232a-n, TPA devices 240a-n, employer devices 238a-n, funding sources 234a-n, or POS terminals 120a-n. The application tier can include the gateway engine 310, the opportunity engine 320, and the scoring engine 330. The application tier can interact with the data tier to obtain the transaction data. The data tier can include data persistence mechanisms (database servers, file shares, etc.) and the data access layer that encapsulates the persistence mechanisms and exposes the data. The data tier can include databases 214. The data tier can include an application programming interface (API) to the application tier. The databases 214 can include stored procedures (e.g., SQL statements) that perform tasks with respect the stored data.

The system 200 can include, access, or otherwise communicate with one or more third-party administrator (“TPA”) devices 240a-n. A TPA can refer to an organization that processes insurance claims or certain aspects of employee benefit plans for a separate entity. A TPA can refer to organizations within the insurance industry which “administer” other services such as Underwriting or Customer Service. In some cases, a TPA can handle the claims processing for employers 238a-n that self-insures its employees 232a-n. Thus, the employers 238a-n are acting as an insurance company and underwrites the risk. The risk of loss can remains with the employer 238a-n, and not with the TPA 240a-n. An insurance company may also use a TPA 240a-n to manage its claims processing, provider networks, utilization review, or membership functions. The TPA 240a-n can handle many aspects of other employee benefit plans such as the processing of retirement plans and flexible spending accounts. Many employee benefit plans have highly technical aspects and difficult administration that can make using a specialized entity such as a TPA 240a-n more cost effective than doing the same processing in house.

In the health care industry, for example, TPAs 240a-n can administer all or a portion of the claims process. TPAs 240a-n can be contracted by a health insurer or self-insuring companies to administer services, including claims administration, premium collection, enrollment and other administrative activities. For example, an employer 238a-n may choose to help finance the health care costs of its employees 232a-n by contracting with a TPA 240a-n to administer many aspects of a self-funded health care plan.

Administrators, such as companies or health insurance providers, can establish electronic benefits accounts such as flexible spending accounts or healthcare tax benefit accounts (e.g., health savings accounts) for participants such as employees, subscribers, or customers. These electronic benefits accounts can provide a tax advantage for the participants. Administrators that establish or provide electronic tax benefits accounts for various participants of those accounts can utilize backend information technology infrastructure to process, analyze, monitor or manage the electronic tax benefits accounts. The tax benefit management information technology infrastructure can be configured with processing rules that are applied to electronic transactions. Electronic transactions can include allocating funds to the tax benefit account, withdrawing funds from the tax benefit account, making a purchase with funds from the tax benefit account, modifying a profile of the tax benefit account, or submitting a claim. The management information technology infrastructure can apply one or more rules to each type of transaction to determine an event. As the types of transactions and rules increase in number and complexity, the types and events can also increase in number and complexity, thereby consuming an increasing amount of resources of the information technology infrastructure. For example, events such as a card denial increases the number of transaction attempts, communications with the server, account resets, profile corruption, or resources consumed by a point-of-sale device initiating the transaction.

The employer devices 238a-n can refer to a device used by an entity or organization that is associated with the participant computing devices 232a-n of the employees of the employer. For example, Employer A can be a software company that has a thousand employees associated with the participant computing devices 232a-n. The employees can obtain health care or other services, and pay for those services at a POS terminal 236a-n of the service provider.

Data packets can be generated by a device 240a-n at an administrator. The device can refer to an administrator device (“administrator device”) such as administrator device 240a-n. The administrator device 240a-n may monitor data from the various electronic benefits accounts associated with the administrator. The accounts associated with the administrator may be accounts that are managed or maintained by the administrator. The administrator may be a point of contact for customers or participants of the associated accounts. The client 102, which may correspond to an individual participant of the administrator's electronic benefits account, may access the account and perform a number of actions with respect to the account, such as, fund the account (e.g., via heterogeneous electronic funding sources 234a-n), withdraw from the account, charge the account, and the like. The administrator of the electronic benefits account, as the caretaker of the account, may adjust parameters associated with the account, such as, monthly fees, minimum running balances, etc. At the same time, the DPS 120 may monitor the data, parameters, and performance of the account and store the information under an administrator profile associated with the administrator of the account. The DPS 120 may receive the data associated with the individual participants and their individual accounts from the client's 102 and the parameter data associated with the accounts from the administrator device 240a-n via the network 104.

An administrator device 240a-n can be the place where an administrator may perform various functions of the administrator, for example, functions associated with electronic benefits accounts of the administrator. The administrator device 240a-n is the point at an administrator that may send requests or transaction information to the DPS 120 for further processing or data collection. The administrator device 240a-n may also be configured to transmit an identifier associated with the administrator corresponding to the administrator device 240a-n for identification by the DPS 120. The receiving of the identifier initiates a fraud detection and control process.

The administrator device 240a-n can include hardware and software. Administrators can utilize scanners, EFTPOS terminals, touch screens and any other wide variety of hardware and software available for use with administrator device 240a-n. For example, an administrator can use software to make adjustments to parameters associated with their electronic benefits accounts.

The administrator device 240a-n can include advanced features to cater to different functionality, such as account participant forecasts and estimates, account simulation, communication with participants of accounts, performing actions associated with individual participant accounts (e.g., freezing an account), collecting data from one or more of the participant accounts, etc., all built into the administrator software. The administrator device 240a-n can be configured to execute user-input commands with respect to the electronic benefits accounts of the administrator.

The communication interface 204 can receive data packets. The data packets can carry one or more electronic transactions. In some cases, the data packets can carry multiple electronic transactions. In some cases, the data packets can be received over a duration of time. The electronic transactions can occur over the duration of time. In some cases, the electronic transaction information carried via the data packets can be received by the DPS 120 in real-time, such as responsive to the occurrence of the electronic transaction. In some cases, the DPS 120 can receive the information about the electronic transactions in a bulk upload or batch upload. Receiving the information about the electronic transaction sin a bulk upload or batch upload can reduce computing resource utilization or network bandwidth usage, thereby improving the efficiency of the DPS 120. For example, the provider of the information about the electronic transactions can compress the information and generate data packets carrying the compressed information in a single batch or bulk transmission, thereby reducing network bandwidth utilization.

The electronic transaction information carried via the data packets can include information that facilitates performance of the electronic transaction, or analyzing the electronic transaction to detect fraudulent activity. The electronic transaction can a source identifier pointing to a data structure storing a resource, a destination identifier corresponding to a data structure to transfer the resource, and an intermediary identifier corresponding to an entity that provides at least a portion of the resource stored in the data structure. The source identifier can refer to an account identifier that contains the resource being transferred from a source to a destination. The source identifier can refer to an account of an employee of an employer. The source identifier can correspond to an account associated with a participant computing device 232. The resource can correspond to an electronic resource or physical resource being represented in an electronic form. The resource can refer to or include a token, currency, points, or other resource that can be transferred from the source to a destination. The destination identifier an correspond to an entity or organization receiving the resource. The destination identifier can correspond to a provider of a service or good that is receiving the resource in return for performing the service or providing the good to the employee. The intermediary identifier can correspond to an entity that stores, holds, manages, provides, or maintains the resource. The intermediary identifier can refer to or correspond to the employer device 238a-n, a heterogeneous electronic funding source 234a-n or TPA 240a-n.

An identifier corresponding to a data structure can refer to or include an identifier pointing to a data structure, such as a memory pointer. The identifier corresponding to a data structure can refer to or include an identifier used by a lookup to retrieve, identify, access or select the data structure. The identifier can label the data structure. The identifier can be mapped to the data structure.

The data packets or electronic transaction can be generated by a device at a merchant to conduct an electronic transaction at the merchant. The device can refer to a point of sale terminal (“POS terminal”) such as POS terminal 236a-n. The POS terminals 236a-n are the devices at which retail transactions are initiated. The POS terminals 236a-n are the points at which a customer of the entity or merchant makes a payment to the merchant in exchange for goods or services. At the point of sale the merchant can calculate the amount owed by the customer and provide options for the customer to make payment. The merchant can also issue a receipt for the transaction.

The POS terminal 236a-n can include hardware and software. Merchants can utilize weighing scales, scanners, electronic and manual cash registers, EFTPOS terminals, touch screens and any other wide variety of hardware and software available for use with POS terminal 236a-n. For example, a pharmacy can use software to customize the item or service sold when a customer has a special medication request.

The POS terminal 236a-n can include advanced features to cater to different functionality, such as inventory management, CRM, financials, warehousing, flexible spending account transactions, etc., all built into the POS software. The POS terminal 236a-n can be configured to conduct a transactions using a debit card, multipurse card, Bluetooth, near field communications, smartphone, smartwatch, mobile telecommunications computing device, wearable communications, RFID, etc.

The DPS 120 can include a communications interface 204. The communications interface 204 can execute on one or more processors of a server. The communications interface 204 can include one or more communications ports and be configured with one or more network protocols. Communications ports can include, e.g., network ports, Ethernet ports, WAN ports, I/O ports, or software ports. The communication port can be configured with a network protocol such as Transport Layer Protocols such as TCP/IP or UDP that are configured to receive and process data packets received via a computer network. The port can include or be associated with an IP address of a host and a protocol type of the communication.

The communications interface 204 can receive data packets generated by the POS terminal 236a-n responsive to an electronic transaction resulting in transmission of a request to adjudicate a single claim against an electronic benefits account. The request to adjudicate a single claim against the electronic benefits account is transmitted responsive to a user swiping a payment card at the POS terminal. The payment card can include identifying information that can be used to identify an account identifier of the electronic benefit account (e.g., source identifier) against which to adjudicate the claim. The data packets can include header information and payload information. Multiple data packets can be strung together in a sequence. The header information can refer to TCP/IP headers that include fields such as source port, destination port, sequence number, acknowledge number, window size, etc. The payload information of the data packet can include information related to the electronic transaction, the request to adjudicate a single claim, the merchant, or the customer. The DPS 120 can receive the data packet with header information and payload information and process the packets to obtain information for further processing. The payload can include data identifying the POS terminal 236a-n (e.g., POS terminal 236a) at which the electronic transaction occurred, the merchant providing the POS terminal 236a, a merchant category of the merchant, financial information associated with the user performing the electronic transaction (e.g., via a card swipe or other communication technique used to perform the electronic transaction), an amount of expenditures of the electronic transaction, and other information facilitating adjudication of the single claim. The data packets (e.g., via the payload) can include the request to adjudicate the single claim. The request can specify the electronic benefits account for adjudication. The request can specify information for identifying a policy for performing the adjudication. The payload can include data identifying a merchant category of the merchant, an electronic benefits account, and a monetary amount of the electronic transaction.

The data packets can carry data identifying a merchant or merchant category of the merchant. The data carried by the data packets include a merchant category code or identifier (e.g., dental, medical, etc.). The data identifies a merchant, and the DPS 120 determines a merchant category based on the identification of the merchant by, for example, using a merchant to merchant category mapping or lookup table stored in database. The data packets carrying the request to adjudicate the single claim against the electronic benefits account include a data structure having a first field indicating a merchant identifier, a second field indicating a total amount of expenditures, and a third field indicating the electronics benefit account. The data packets can be generated by a merchant device (e.g., a client device 102 of a merchant) to conduct an electronic transaction at the merchant, and the data packets carry data identifying a merchant category of the merchant, the electronic benefits account maintained and configured on the DPS 120, and a total monetary amount of the electronic transaction.

The data packets (e.g., payload of the data packets) can further identify an electronic account maintained and configured on the server. The electronic account can be maintained and configured in a database 214. The electronic account can correspond to a user and have a unique identifier. The unique identifier can include numbers, letters, characters, symbols, etc. The electronic account can be associated with the customer making the transaction at the merchant. The POS terminal 236a can receive or determine the electronic account identifier via a card swipe or other communication technique employed at the POS terminal 236a, which the POS 236a can then convey to the DPS 120.

The communications interface 120 can further receive data packets (e.g., payload information) identifying a monetary amount of the electronic transaction, such as a total amount of expenditures. The monetary amount can be for the purchase of goods or services made at the merchant. The monetary amount of the transaction can refer to the amount of funds in consideration for goods or services obtained from the entity or merchant. The merchant or entity can refer to the entity at which a point-of-sale terminal or device used to make the transaction is located or with which the terminal is associated. The monetary amount can be in any currency (e.g., United States dollars) or units. The monetary amount can be further tied to a category, such as medical services.

The POS terminal 236a can generate multiple data packets for a single transaction. The multiple data packets can each include a header and a payload. The header can indicate that the multiple data packets can be to be grouped together for routing, transmission or processing purposes.

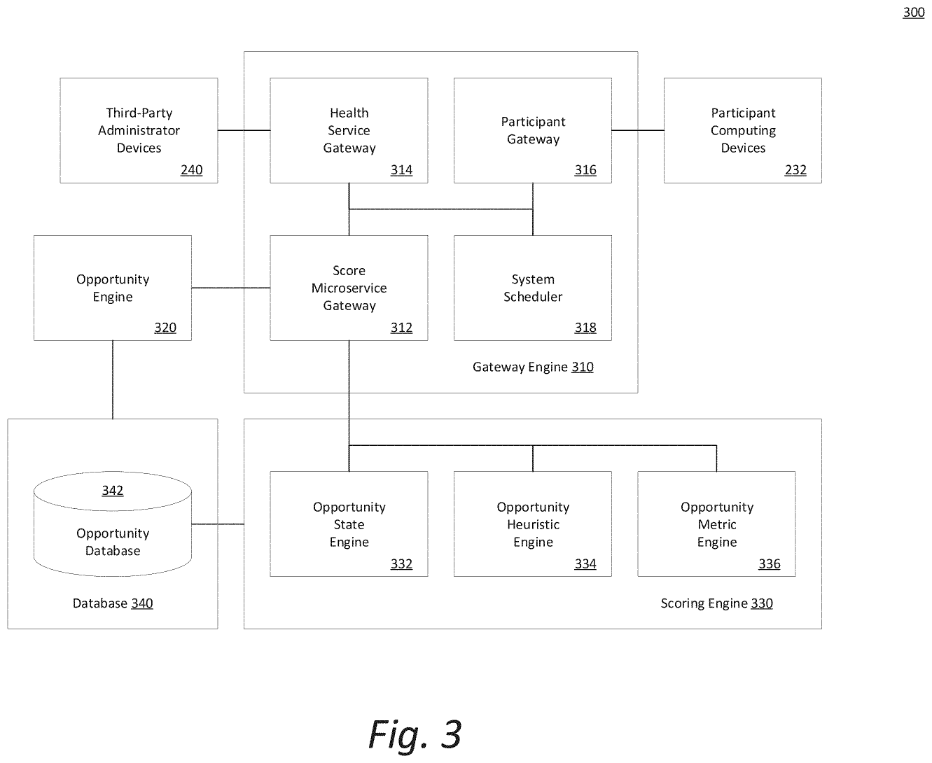

FIG. 3 illustrates an example data processing system, in accordance with present implementations. The example data processing system (DPS) 300 corresponds to DPS 120. The DPS 300 includes at least one of the gateway engine 310, the opportunity engine 320, the scoring engine 330, and the database 340. The gateway engine 310 includes score microservice gateway 312, a health service gateway 314, a participant gateway 316, and a system scheduler 318. The scoring engine 330 includes an opportunity state engine 332, an opportunity heuristic engine 334, and an opportunity metric engine 336.

The gateway engine 310 can generate, modify, block, transmit, and the like, communication messages between at least the participant computing devices 232, the third party administrator device 240, the opportunity engine 320, the scoring engine 330 and the database 340. The gateway engine 310 includes or is associated with one or more operating systems, virtual machines, or interpreters, for example, to generate, modify, block, or transmit, for example, the communication messages. The communication messages can be or include commands formatted as at least one GET request, or PUT request, for example. The gateway engine 310 includes one or more logical or electronic devices including but not limited to integrated circuits, logic gates, flip flops, gate arrays, programmable gate arrays, and the like. It is to be understood that any electrical, electronic, or like devices, or components associated with the gateway engine 310 can also be associated with, integrated with, integrable with, replaced by, supplemented by, or complemented by, for example, a system processor or any component thereof.

The score microservice gateway 312 can restrict, regulate, modify, block, or transmit, for example, one or more communication messages according to one or more transmission criteria or security criteria. The score microservice gateway 312 enforces one or more security policies including one or more security criteria. The score microservice gateway 312 applies a first security policy to communication messages associated with the participant communication devices 232. The first security policy is or includes a user level trust zone, associated with a low trust level. The score microservice gateway 312 applies a second security policy to communication messages associated with the third party administrator devices 240. The second security policy is or includes a third party level trust zone, associated with a low trust level. The score microservice gateway 312 applies a third security policy to communication messages associated with the gateway engine 310 and the opportunity engine 320. The third security policy is or includes a cloud trust zone, associated with a medium trust level. It is to be understood that various security policies and various trust zones include, but can be not limited to varying numbers and degrees of security encapsulation for example. The security encapsulation includes token validation, password validation, hardware key validation, symmetric encryption key validation, and asymmetric encryption key validation.

The health service gateway 314 can generate one or more communication messages compatible with one or more of the third party administrator devices, based on communication messages received from the score microservice gateway 312. The health service gateway 314 is further operable to generate one or more communication messages compatible with the score microservice gateway 312, based on communication messages received from one or more of the third party administrator devices 240. The health service gateway is or includes at least one application programming interface (API) compatible with the third party administrator devices 240 and the score microservice gateway 312. The health service gateway 314 is or includes at least one API to interface with third party administrator devices 240 associated with multiple third party administrator services. As one example, a third party administrator service can be a participant's health insurance service or a participant's prescription drug records service. The second security policy is configured to securely couple the gateway engine 310 to the third party administrator devices by the health service gateway 314, in accordance with one or more health records standards. As one example, health records standards may be associated with Health Insurance Portability And Accountability Act (HIPAA) requirements for example.

The participant gateway 316 can generate one or more communication messages compatible with one or more of the participant computing devices 232, based on communication messages received from the score microservice gateway 312. The participant gateway 316 is further operable to generate one or more communication messages compatible with one or more of the participant computing devices 232, based on communication messages received from the score microservice gateway 312. The participant gateway 316 is or includes at least one API compatible with participant computing devices 232 and the score microservice gateway 312. The participant gateway is configured to encapsulate at least one opportunity object received from the opportunity engine and transmit the encapsulated opportunity object to one or more of the participant computing devices 232. The participant gateway 316 encapsulates an opportunity object in a JSON container, an encrypted package, or a compressed package, for example. The participant gateway 316 transmits the opportunity object or the encapsulated opportunity object in accordance with the first security policy.

The system scheduler 318 can generate, modify, block, transmit, and the like, communication messages associated with opportunity objects in accordance with at least one transmission schedule or at least one batch processing criterion. The transmission schedule is a synchronous transmission schedule including at least one transmission interval between transmission times. The gateway engine 310 transmits, or generates, for example, one or more communication messages associated with opportunity objects at a transmission time. In some implementations, a batch processing criterion includes a connectivity condition between at least one of the participant computing devices 232 and one or more of the gateway engine 310 and the DPS 120. The system scheduler 318 enters an asynchronous mode in response to determining that connectivity with one or more particular participant computing devices 240 is below a connectivity threshold. The batch processing criterion includes the connectivity threshold. As one example, a connectivity threshold can be a minimum lag, bandwidth, or uptime, for example associated with a minimum ability to transmit one or more opportunity objects. The system scheduler 318 includes one or more queues to asynchronously store, buffer, for example any communication messages that cannot be transmitted at a particular transmission time due to failure to satisfy a connectivity threshold or satisfying a batch processing criterion. The system scheduler 318 is selectably configurable into a synchronous mode, an asynchronous mode, or both. The system scheduler 318 is selectably configurable by at least one API associated therewith.

The opportunity engine 320 can discover, audit, execute, and the like, one or more opportunity objects. The opportunity engine 320 is operatively coupled to the opportunity database 342 to store, retrieve, modify, generate, link, delete, and the like, one or more objects associated therewith. The opportunity engine 320 is operatively coupled to one or more of the third-party administrator devices 240 by the gateway engine 310. The opportunity engine 320 includes one or more logical or electronic devices including but not limited to integrated circuits, logic gates, flip flops, gate arrays, programmable gate arrays, and the like. It is to be understood that any electrical, electronic, or like devices, or components associated with the opportunity engine 320 can also be associated with, integrated with, integrable with, replaced by, supplemented by, or complemented by, for example, a system processor or any component thereof. The opportunity engine 320 interacts with, modifies, or obtains, for example, at least one participant object. In some implementations, a participant object is configured to encapsulate at least one executable, module, link, and the like associated with a participant of a health support system and the like. The participant object includes at least one object identifier, timestamp, participant key, geolocation monitor, insurance linker, prescription linker, pharmacy linker, participant profile linker, and opportunity object linker.

The scoring engine 330 can score, modify, validate, and the like, one or more opportunity objects or components thereof. The scoring engine 330 is operatively coupled to the opportunity database 342 to store, retrieve, modify, generate, link, delete, and the like, one or more objects associated therewith. The scoring engine 330 is operatively coupled to one or more of the third-party administrator devices 240 by the gateway engine 310. The scoring engine 330 includes one or more logical or electronic devices including but not limited to integrated circuits, logic gates, flip flops, gate arrays, programmable gate arrays, and the like. It is to be understood that any electrical, electronic, or like devices, or components associated with the scoring engine 330 can also be associated with, integrated with, integrable with, replaced by, supplemented by, or complemented by, for example, a system processor or any component thereof. The scoring engine 330 includes at least one of an opportunity state engine 332, an opportunity heuristic engine 334, and an opportunity metric engine 336.

The opportunity state engine 332 can detect, monitor, obtain, and the like, at least one system characteristic associated with at least one opportunity object or participant computing device performance metric. The opportunity scoring engine can monitor a state of one or more system characteristic. In some implementations, a system characteristic is or is associated with at least one of an opportunity object, an electronic device among the participant computing devices 232 associated with the participant or the opportunity object, a health support account associated with the participant, a financial account associated with the participant, and a medical records account associated with the participant.

The opportunity heuristic engine 334 can validate an opportunity object based on one or more validation factors. The opportunity heuristic engine 334 can validate an opportunity object based on one or more metrics associated with a participant profile, a service or account associated with the participant, a device associated with the participant, a physical state of a participant, a physical state of a device associated with the participant, and the like. The opportunity heuristic engine 334 can conduct one or more validation or integration operations based on a change in state detected at the opportunity state engine 332. The opportunity heuristic engine 334 can validate one or more opportunity objects in response to a scheduling process, batch process, selection process from a participant computing device 232, and the like. As one example, the opportunity heuristic engine can validate whether an opportunity presented to a participant computing device is or remains valid at a time, place, or participant condition of selection of the opportunity. The opportunity heuristic engine 334 can integrate a plurality validation factors, outputs from validators, and the like, to validate one or more opportunities associated with a participant.

The opportunity metric engine 336 can generate, or modify, for example, at least one performance metric associated with at least one opportunity object or participant computing device. The opportunity metric engine 336 can generate, or modify, for example, at least one aggregate performance metric associated with a participant, a participant score, a participant point value, and the like. The opportunity metric engine 336 can generate, or modify, for example, at least one performance metric associated with at least one opportunity object. As one example, the opportunity metric engine 336 can modify a point value, or score value, for example, associated with an opportunity object based on a time of selection. In this example, the opportunity metric engine can reduce a point value, or score value, for example if a selection is received outside of a predetermined time threshold. As another example, the opportunity metric engine 336 can modify an availability, interactability, or visibility, for example, associated with an opportunity object based on a time threshold for selection. In this example, the opportunity metric engine can block an opportunity object not satisfying a predetermined time threshold. In some implementations, availability includes, but is not limited to, visibility on, communication to, or authorization to communicate to, or visibility, for example, an electronic device among a plurality of participant computing devices 232. In some implementations, interactability includes, but is not limited to, modification of a gateway to obtain, communicate, or respond to, or visibility, for example, a selection of or interaction with an opportunity object. The selection of or interaction with an opportunity object includes a selection or interaction obtained from or at an electronic device among a plurality of participant computing devices 232.

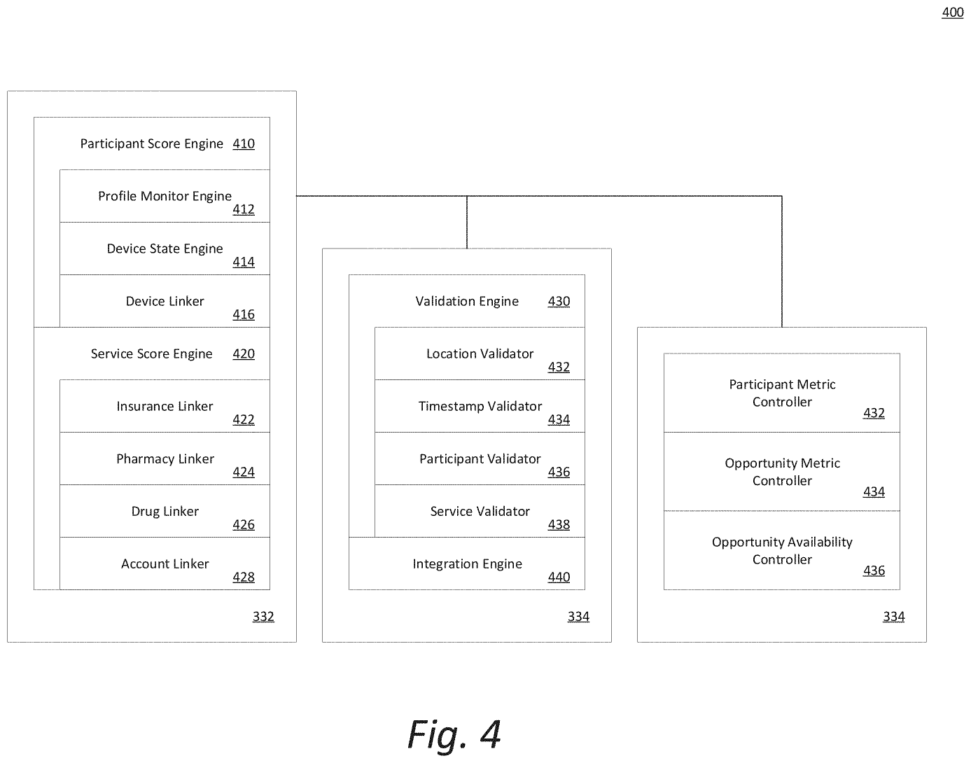

FIG. 4 illustrates an example scoring engine further to the example data processing system of FIG. 3, in accordance with present implementations. In some implementations, an example scoring engine 400 corresponds to the scoring engine 330. The scoring 400 includes at least one of the opportunity state engine 332, the opportunity heuristic engine 334, and the opportunity metric engine 336. In some implementations, at least one of the example scoring engine 440 and the opportunity state engine 332 includes a participant score engine 410 and a service score engine 420. In some implementations, at least one of the example scoring engine 440 and the opportunity heuristic engine 334 includes a validation engine 430 and an integration engine 440. In some implementations, at least one of the example scoring engine 440 and the opportunity metric engine 336 includes a participant metric controller 432, an opportunity metric controller 434, and an opportunity availability controller 436.

The participant score engine 410 can detect, obtain, identify, or select, for example, at least one state associated with a performance metric and a participant. The participant score engine 410 can identify one or more states corresponding to one or more performance metric values, operations, or changes, for example. As one example, the participant score engine 410 can detect a chance in a participant profile to indicate, or initiate, for example, a modification, or validation, for example of a performance metric associated with the participant. Thus, in this example, the participant score engine 410 can perform operation in response to a state of a participant profile, device, and the like. The participant score engine 410 includes a profile monitor engine 412, a device state engine 414, and a device linker 416. The participant score engine 410 is operable in accordance with a scheduled updated initiated by a scheduling process or a batching process.

The profile monitor engine 412 can monitor, detect, obtain, identify, or select, for example, at least one state associated with a participant profile object associated with the opportunity object. In some implementations, a participant profile object is configured to encapsulate at least one executable, module, link, and the like associated with a health metric, consumer metric, diagnostic assessment, prognostic assessment, health device interface, for example, associated with a participant object or a participant associated with the participant object. The participant profile object includes an object identifier and a diagnostic encapsulator. The object identifier contains one or more characteristics associated with a pharmacy associated with the participant object. The object identifier 44 contains or includes one or more blocks, links, or executables, for example associated with at least one of the participant object or a participant associated with the participant object. The profile monitor engine can receive a signal, interrupt, or trigger, for example in response to change in state of one or more components of the object identifier or the diagnostic encapsulator. As one example, the profile monitor engine 412 can detect a change in a participant personal identification information including address, phone, and the like.

The device state engine 414 can monitor, detect, obtain, identify, or select, for example, at least one state associated with a participant computing device 232 associated with a particular participant. The device state engine 414 can detect one or more hardware states of an electronic communication device including a smartphone, table, and the like. The device state engine 414 can detect a state of the participant computing device 232 associated with a particular participant in response to a scheduling process, a push notification process, or a polling process, for example. As one example, the device state engine 414 can receive a change in state of a participant smartphone device in response to an activation of geolocation hardware, or software, for example, associated or integrated with the participant smartphone device.

The device linker 416 contains a dynamic link to the participant computing device 232 associated with a particular participant. The device linker 416 can associate the participant with the participant computing device 232 by the device linker 416. The device linker 416 can obtain a device identifier associated with the participant computing device 232. The device identifier includes a MAC address, serial number, firmware identifier, operating system identifier, authentication token, authentication key, participant token, participant key, communication provider identifier, and the like. The device linker 414 can receive, or request, for example, a device state from the participant computing device 232 by the participant gateway 316. The device liner 414 is linked to the participant computing device 232 by a participant computing device identifier generated, or controlled, for example, by the participant gateway 316. As one example, the device linker 416 can identify the participant computing device 232 by an identifier code associated therewith and generated by the participant gateway 316.

The service score engine 420 can detect, obtain, identify, or select, for example, at least one state associated with a performance metric and a third party system associated with a participant. The service score engine 420 is associated with one or more of a an insurance provider system, a pharmacy system, a pharmaceutical records system, and a financial account system. The service score engine 420 includes an insurance linker 422, a pharmacy linker 424, a drug linker 426, and an account linker 428. The service score engine is operable in accordance with a scheduled updated initiated by a scheduling process or a batching process.

The insurance linker 422 contains a dynamic link to an insurance object associated with the participant. The insurance linker 422 can monitor, generate, or store, for example, a link, or reference, for example to at least one insurance provider record associated with the participant. The insurance linker 422 can detect one or more configuration states of associated with at least one insurance provider record. The insurance linker 422 can detect a state, type, value, availability, of the like, associated with insurance provider record. As one example, the insurance linker 422 can receive a change in state of an insurance provider record in response to an activation of a health service, or reference, for example, with the participant smartphone device. As another example, the insurance linker 422 can detect or flag an insurance provider record based on type, or tier, or reference, for example, of the record. The insurance linker 422 is operable in response to a scheduling process, a push notification process, or a polling process, or reference, for example.

The pharmacy linker 424 can monitor, generate, or store, for example, a link, or reference, for example to a pharmacy object associated with the participant. The pharmacy linker 424 can detect one or more configuration states of associated with at least one pharmacy record associated with the participant. The pharmacy linker 424 can detect a state, type, value, availability, of the like, associated with pharmacy record. As one example, the pharmacy linker 424 can receive a change in state of pharmacy record in response to selection of a particular preferred or available pharmacy entity by, at or in communication with the participant smartphone device. The pharmacy linker 424 is operable in response to a scheduling process, a push notification process, or a polling process, or reference, for example.

The drug linker 426 can monitor, generate, or store, for example, a link, or reference, for example to a drug object associated with the pharmacy linker. The drug linker 426 generates a link, or reference, for example to at least one drug object within a prescription associated with a participant. As one example, the drug linker 426 can generate and maintain a link to a drug object statically or dynamically based on a drug provider by a health support service, or support service, for example. In this example, the drug linker 426 can monitor the drug object associated with the participant for any changes to the drug composition, availability, price, discount to price, and the like. The drug linker 426 is operable in response to a scheduling process, a push notification process, or a polling process, for example.

The account linker 428 contains a dynamic link to a financial account associated with the participant. The account linker 428 can monitor, generate, or store, for example, a link, or reference, for example to at least one financial account, financial account, interface, or financial account query system, for example, associated with the participant object. The account linker 428 can detect one or more configuration states of associated with at least one financial account associated with the participant. As one example, the account linker 428 can detect at least one of a direct deposit state, a recurring contribution state, a recurring contribution amount, a minimum funding amount, a maximum funding amount, a minimum transfer amount, a maximum transfer amount, a routing number, an account number, a security token, a security password, a security key, a participant key, and the like. The account linker 428 can detect a state, type, value, availability, of the like, associated with financial account. As one example, the account linker 428 can receive a change in state of an insurance provider record in response to an activation of a health service, for example, with the participant smartphone device. As another example, the account linker 428 can detect or flag linked financial account based on type, or tier, for example, of the account. In this example, the account linker 428 can detect whether a financial account is a health savings account (“HSA”). The account linker 428 is operable in response to a scheduling process, a push notification process, or a polling process, for example.

The validation engine 430 can validate, evaluate, filter, analyze, or compare for example one or more states, configurations, and the like associated with opportunities and participants. The validation engine 430 can obtain one or more response from at least one of the participant engine 410, the service score engine 420, and any component thereof. The validation engine 430 obtains one or more threshold, ideal, control, limit, or like values associated with a predetermined condition, state, or heuristic for example. The validation engine 430 obtains one or more current conditions, states, or configurations for example for validation against a predetermined heuristic for example. As one example, the validation engine 430 can obtain a current state from the opportunity state engine 332 or any component thereof, and a predetermined heuristic from the opportunity heuristic engine 430 or any component thereof. As another example, the validation engine 430 can obtain the predetermined heuristic from one or more of the validators 432, 434, 436 and 438. The validation engine 430 includes a location validator 432, a timestamp validator 434, a participant validator 436, and a service validator 438. In some implementations, validation engine 430 is operable in accordance with a scheduled updated initiated by a scheduling process or a batching process, or an asynchronous process initiated by the opportunity state engine 332 or the gateway engine 310.

The location validator 432 can validate a target location associated with the opportunity object and the participant object against a location heuristic. The target location is or includes one or more of a physical address, postal address, geolocation, coordinate pair, or spherical coordinate, for example. The location heuristic is or includes one or more of a maximum distance, a minimum distance, a maximum travel time, a minimum travel time, a maximum resource expenditure to travel, a minimum resource expenditure to travel, and the like. In some implementations, a resource expenditure is or includes at least one of an amount of fuel, a cost of fuel, a cost for road usage, a cost for transit usage, a vehicle rental cost, and the like. The location validator contains the location heuristic. The location validator 432 obtains a target location from a participant computing device 240 by at least one of the gateway engine 310 and score microservice gateway 312. As one example, the location validator 432 can obtain a target location from the pharmacy linker 424. In this example, the target location contains one or more addresses, pharmacy location codes, phone numbers, and the like associated with a pharmacy linked by the pharmacy linker 424. As another example the target location can include a pharmacy geolocation, one or more latitude coordinates, longitude coordinates, spherical coordinates, Global Positioning System (“GPS”) coordinates, pathfinding routes, and the like. The location validator 432 is operable in accordance with a scheduled updated initiated by a scheduling process or a batching process, or an asynchronous process initiated by the opportunity state engine 332 or the gateway engine 310.

The timestamp validator 434 can validate a target timestamp associated with the opportunity object and the participant object against a timestamp heuristic. The target location is or includes one or more of a UNIX timestamp, a date and time, and the like. The timestamp heuristic includes at least one of an expiration timestamp, an activation timestamp, and the like. As one example, the target timestamp is or includes a timestamp at a time of selection of an opportunity at or in communication with a participant computing device 232. As another example, the timestamp heuristic can include at least one timestamp at which time an opportunity object ceases to remain available for selection by a participant. As another example, the target timestamp can include at least one timestamp at which time an opportunity starts to become available for selection by a participant.

The participant validator 436 can validate a participant object and a participant profile object associated with the opportunity object against a participant heuristic. The participant validator 436 can authenticate a participant object or a participant associated with a participant object by a participant key. The participant key contains a key based on multiple identifiers associated with a participant. The participant key is based on at least one of an account code, an employer identifier and an employee identifier associated with the participant and the health service associated with the participant. The participant key is encrypted, or hashed, for example. The participant validator determines whether a participant object satisfies a particular participant metric. The participant metric includes at least a support service, or support account, for example associated with the participant object. The participant heuristic includes at least a medical history, prescription history, current medical conditions, family medical history, evaluation charts, and the like associated with a participant object or a participant associated with the participant object and compatible with a particular opportunity object. The participant validator 436 is configured to modify, obtain, transmit, or store, for example, information associated therewith or contained therein in accordance with one or more diagnostic security protocols. As one example a diagnostic security protocol includes a restriction on access, or decryption, for example, based on a HIPAA or like restriction. The participant validator 436 is operable in accordance with a scheduled updated initiated by a scheduling process or a batching process, or an asynchronous process initiated by the opportunity state engine 332 or the gateway engine 310.

The service validator 438 can validate one or more of an insurance object, a drug object, and a prescription object associated with the opportunity object against a service heuristic. The service heuristic includes a type of support service, or support account, for example associated with the participant object. As one example, the participant validator 436 can determine whether a financial account associated with the participant validator 436 is an HSA account, where the service heuristic is a type of financial account. The service validator 438 can validate associate of at least one quantity, metric, volume, amount, mass, time, dosage, and the like of at least one drug object with a particular opportunity object or participant object, where the service heuristic is an availability or promotional metric associated with the drug object. The service validator 438 is configured to encapsulate at least one personally-identifiable health record within a secure wrapper. The service validator 438 is operable in accordance with a scheduled updated initiated by a scheduling process or a batching process, or an asynchronous process initiated by the opportunity state engine 332 or the gateway engine 310.

The integration engine 440 can perform one or more aggregated validation operations including validation by one or a plurality of the location validator 432, the timestamp validator 434, the participant validator 436, and the service validator 438. The integration engine 440 can validate an opportunity object or a participant object in accordance with validation logic including input from one or more of the location validator 432, the timestamp validator 434, the participant validator 436, and the service validator 438. The integration engine 440 can apply one or more logical, arithmetic, algebraic, combinatoric, or like operations to generate a validation for an opportunity object or a participant object. As one example, the integration engine 440 can validate an opportunity that satisfies both a location heuristic and a timestamp heuristic. As another example, the integration engine 440 can validate an opportunity that satisfies a timestamp heuristic and at least one of a participant heuristic and a service heuristic. Thus, The integration engine 440 can communicate with the validation engine 330 to perform complex validation operations therewith. The integration engine 440 can receive one or more integration profiles including one or more integration instructions executable by the integration engine 440. In some implementations, integration engine 440 is operable in accordance with a scheduled updated initiated by a scheduling process or a batching process, or an asynchronous process initiated by the opportunity state engine 332 or the gateway engine 310.

The participant metric controller 432 can modify a participant metric associated with a particular participant object. The participant metric controller 432 can aggregate one or more metrics, scores, or values, for example associated with one or more validations conducted by the validation engine 430 and the integration engine 440. As one example, the participant metric controller 432 can add an opportunity metric, score, or value, for example associated with the opportunity to a participant object, upon receiving an indication of a selection of the opportunity object at a participant computing device associated with the participant object. The participant metric controller 432 can subtract an opportunity metric, score, or value, for example associated with the participant object, upon receiving an indication of a change in state of the participant object. As one example, the participant metric controller 432 can reduce a participant metric upon indication that a participant computing device ceases sharing its location or allowing its location to be shared with the location validator 432. Thus, The participant metric controller 432 can manage an aggregate participant metric, score, or value, for example individualized to a particular participant object. The participant metric controller 432 is operable in accordance with a scheduled updated initiated by a scheduling process or a batching process, or an asynchronous process initiated by the opportunity state engine 332 or the gateway engine 310.

The opportunity metric controller 434 can modify an opportunity metric associated with a particular opportunity object. The opportunity metric controller 434 can aggregate one or more metrics, scores, or values, for example associated with one or more validations conducted by the validation engine 430 and the integration engine 440. As one example, the opportunity metric controller 434 can generate an opportunity metric, score, or value, for example associated with an opportunity object based on satisfying a location heuristic and a service heuristic. In this example, the opportunity metric controller can generate a score adding a point value associated with the location heuristic and a second point value associated with the service heuristic. Thus, The opportunity metric controller 434 can generate an individualized high value opportunity incorporating validation from diverse sources including an individual participant computing device and a third party administrator device. The opportunity metric controller 434 is operable in accordance with a scheduled updated initiated by a scheduling process or a batching process, or an asynchronous process initiated by the opportunity state engine 332 or the gateway engine 310.

The opportunity availability controller 436 can modify whether to display, select, or execute, for example, an opportunity object associated with a particular participant object. The opportunity availability object can modify whether to display, select, or execute, for example, an opportunity object in realtime or substantially realtime in communication with one or more secure third party support services, participant services, support accounts, and the like. As one example, the opportunity availability controller 436 can cease to display an opportunity object in realtime when the opportunity object fails to satisfy a location heuristic. In this example, the opportunity object can obtain a validation result from the location validator repeatedly in realtime to modify availability of the opportunity object. As another example, the opportunity availability controller 436 can cease to display an opportunity object in realtime when the opportunity object fails to satisfy a timestamp heuristic. In this example, the opportunity availability controller 436 can cease to display the opportunity object upon expiration of the opportunity object as determined by the timestamp validator 434. It is to be understood that the opportunity availability controller 436 can obtain realtime validation results from the validation engine 430 and any component thereof, and the integration engine 440. The opportunity availability controller 436 is operable in accordance with a scheduled updated initiated by a scheduling process or a batching process, or an asynchronous process initiated by the opportunity state engine 332 or the gateway engine 310.

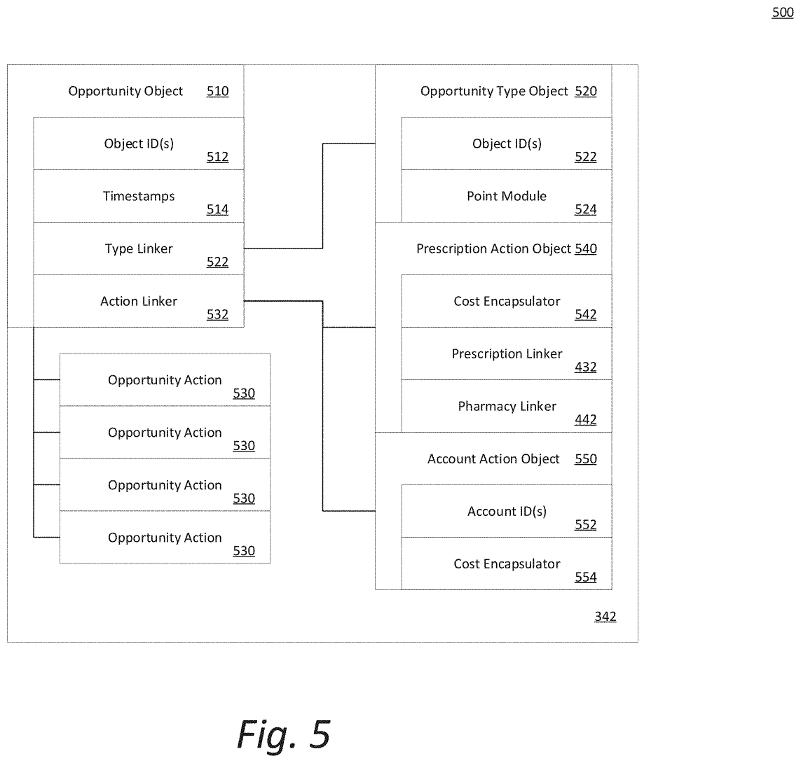

FIG. 5 illustrates an example opportunity database system further to the example DPS of FIG. 3, in accordance with present implementations. The example opportunity database system 500 corresponds to the opportunity database 342. The example opportunity database system 500 includes at least one of an opportunity object 510, an opportunity type object 520, an opportunity action 530, a prescription action object 540, and an account action object 550.

The opportunity object 510 is configured to encapsulate at least one executable, module, link, and the like associated with a participant of a health support system and the like. The opportunity object 510 is associated with, or represents, or execute, for example, characteristics, variables, or executables, or execute, for example associated with a health support account, or a support account, or execute, for example. The opportunity object 510 is configured to execute to facilitate execution of one or more instructions to a health support account, or support account, for example. The opportunity object 510 is configured to execute a financial transaction across a plurality of secure and heterogeneous database system or commercial computing systems. As one example, the opportunity object 510 is configured to execute a transaction with a pharmacy system to obtain a pharmaceutical prescription, including secure patient information and secure financial information of a participant associated with a participant object 410. In this example, the opportunity object 510 achieves the technical solution of secure digital communication across multiple secure systems involving both financial security and patient care security protocols. The opportunity object 510 includes an object identifier 512, a type linker 522, and an action linker 532. The object identifier 512 contains one or more characteristics associated with the opportunity object 510. The object identifier 512 contains or includes one or more blocks, links, executables, for example associated with opportunity object 510.

The type linker 522 contains a dynamic link to the opportunity type object 520. The type linker 522 can generate, or store, for example, a link, or reference, for example to the opportunity type object 520 associated with the opportunity object 510. The type linker 522 associates the opportunity object 510 with the opportunity type object 520 in accordance with one or more of an import process of participant information from one or more third party administrator devices 240, or a selection, or interaction, for example received from a participant computing device 232.

The action linker 532 contains a dynamic link to the opportunity action object 530. The action linker 532 can generate, or store, for example, a link, or reference, for example to one or more opportunity action objects 530 associated with the opportunity object 510. The action linker 532 associates the opportunity object 510 with the opportunity action object 530 in accordance with an import process of participant information from one or more third party administrator devices 240. The action linker 532 associates the opportunity object 510 with the opportunity action object 530 in accordance with a selection, or interaction, for example received from a participant computing device 232. The action linker 532 associates the opportunity object 510 with the opportunity action object 530 in accordance with a scheduled updated initiated by a scheduling process or a batching process.

The opportunity type object 520 is configured to encapsulate at least one executable, module, link, and the like associated with a participant of a health support system and the like. The opportunity object 510 is associated with, or represents, for example, characteristics, variables, or executables, for example associated with an opportunity. As one example, the opportunity type object includes at least one value, metric, executable, or link, for example associated with a time-sensitive opportunity. The opportunity type object 520 includes an object identifier 522 and a point module 524. The object identifier 522 contains one or more characteristics associated with the opportunity type object 510. The object identifier 522 contains or includes one or more blocks, links, or executables, for example associated with opportunity type object 522.

The point module 524 is configured to encapsulate at least one executable, module, link, and the like associated with an execution value of an opportunity object associated with the opportunity type object 520. As one example, the point module 524 includes at least one value, metric, executable, or link, for example associated with a quantitative point value for executing the opportunity object 510. As another example, the point module contains, stores, or is associated with an integer, a whole number, a real number, an algebraic quantity, a logical quantity or the like. In this example, the point module can store a positive number indicating a number of points associated with the opportunity object. The quantitative point value dynamically changes based on one or more opportunity or participant metrics, factors or the like. As one example, the quantitative point value decreases toward zero as a current timestamp approaches a timestamp threshold. The opportunity metric controller 434 can generate, modify, aggregate, subtract, and the like, any metric, value, score, or the like stored at or associated with the point module 524. It is to be understood that a point value or the like associated with the point module 524 and the opportunity object can correspond in type, kind, value and the like to a point value, metric, score and the like associated with at least a participant object, a participant profile object, an insurance object, a pharmacy object, a prescription object, and a drug object.

The opportunity action object 530 is configured to encapsulate at least one executable, module, link, and the like to execute or facilitate execution of at least one action based on at least one of the prescription action object 540 and the account action object 550. The opportunity action object 530 includes one or more instructions, restrictions, security policies, interfaces, and the like to execute or facilitate execution of at least one of the prescription action object 540 and the account action object 550. The opportunity action object 530 include an action associated with an application programming interface (“API”) of the DPS 120. The opportunity action objects is associated with an API call through one or more gateways of the DPS 120. The API call can be a call to conduct actions including SwitchtoDirectDeposit, SetUpMedicineCabinet, ChangeElectronicDelivery, AttachInsuranceCarrier, AddMobilePhone, SignUpForAlerts, SignUpForElectronicTaxForms, and EnableLocationTracking.

The prescription action object 540 is configured to encapsulate at least one executable, module, link, and the like to execute or facilitate execution of at least one action based on the opportunity action object 540. The prescription action object 540 includes one or more instructions, restrictions, security policies, interfaces, and the like to modify the state of at least one medical records system associated with the prescription action object 540. As one example, a medical records system can include a pharmacy system, a health support system, a health insurance system, and the like. The prescription action object 540 include a cost encapsulator 542, the prescription linker 432, and the pharmacy linker 442.

The cost encapsulator 542 is configured to encapsulate at least one executable, module, link, and the like associated with at least one financial value, metric, or the like associated with the prescription object 430. As one example, the cost encapsulator 542 is or includes at least one of a price, pricing table, pricing modifier, and the like. The cost encapsulator 542 is configured to generate at least one modification to a cost value, metric, or the like. As one example, the cost encapsulator 542 is configured to generate a LongtermSavings value indicating a difference between cost for a prescription upon execution of an opportunity as compared to not executing the opportunity. It is to be understood that the cost encapsulator 542 is configured to achieve the technical solution of generating the LongtermSavings value based on multiple factors including multiple drugs that may be subject to cost modification based on multiple prescriptions from multiple health support systems and multiple pharmacy systems. The cost encapsulator 542 is configured to generate the LongtermSavings value with respect to a plurality of participants based on individualized records.

The account action object 550 is configured to encapsulate at least one executable, module, link, and the like to execute or facilitate execution of at least one action based on the opportunity action object 540. The account action object 550 includes one or more instructions, restrictions, security policies, interfaces, and the like to modify the state of at least one financial account system associated with the account action object 550. As one example, a financial account system can include a bank account, an HSA account, a flexible spending account (“FSA”) and the like. The account action object 550 includes an account identifier 552 and a cost encapsulator 554. The account action object 550 includes an account identifier 552 and a cost encapsulator 554. The account identifier 552 contains one or more characteristics associated with the financial account associated with the opportunity action object 530. The account identifier 552 contains or includes one or more blocks, links, or executables, for example associated with a financial account system. As one example, the account identifier 552 is or encapsulates a financial account number, a financial account routing number, a financial account authorization code, a financial account authorization token, a financial account or link, for example.

The cost encapsulator 554 is configured to encapsulate at least one executable, module, link, and the like associated with at least one financial value, or metric, or link, for example associated with the account identifier 552. As one example, the cost encapsulator 542 is or includes at least one of a financial contribution amount, financial payment amount, financial credit amount, or financial debit amount, or link, for example associated with the account identifier 552. The cost encapsulator 554 is configured to generate at least one modification to a cost value, or metric, or link, for example associated with the account identifier 552. As one example, the cost encapsulator 542 is configured to generate a MaxOutContributions value instructing a financial account system to increase a payment to the financial account according to one or more threshold criteria contained in the account identifier 552. It is to be understood that the cost encapsulator 554 is configured to achieve the technical solution of generating the MaxOutContributions value based on secure direct communication with a financial account system.

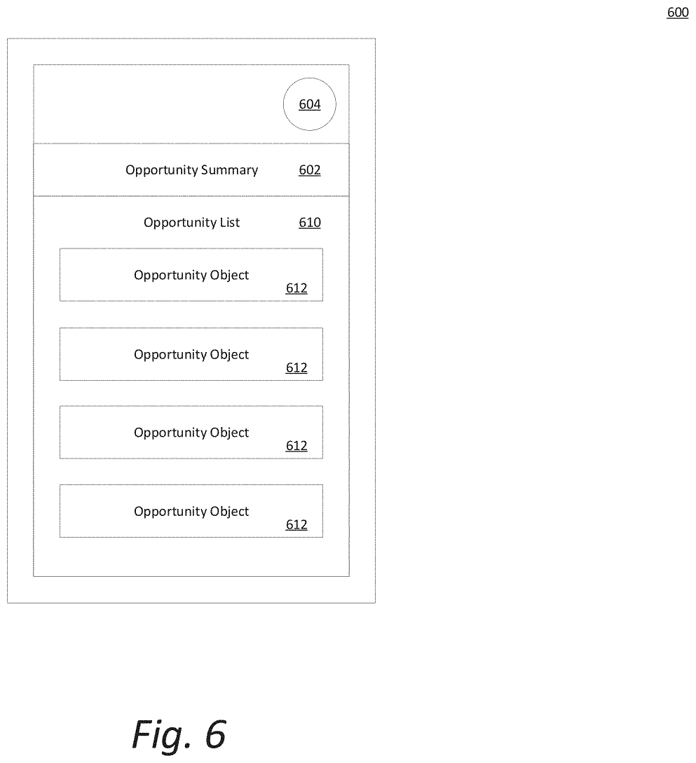

FIG. 6 illustrates an example electronic device associated with an example DPS, in accordance with present implementations. The example electronic device 600 includes a processor, memory device, and display device to generate a graphical user interface, and a communication interface to communication with the data processing system as discussed above with respect to FIGS. 1A-D and 2. The example electronic device includes an opportunity summary region 602, a score summary region 604, an account action object region 606, and an opportunity list region 610. The example electronic device 600 can generate a graphical user interface (“GUI”) representing, displaying, and the like, one or more of objects of FIGS. 4 and 5. In some implementations, The example electronic device 600 is or includes a GUI associated with a mobile operating system. In some implementations, The example electronic device 600 is further operable to receive selection, input, and the like from a touch-based input system disposed thereon, therewith, or link, for example. The touch-based input system is a capacitive or a resistive touch interface. That the mobile interface includes a presentation device including but not limited to an LCD, LED, or OLED or link, for example.

The opportunity summary region 602 is configured to present at least one metric associated with one or more opportunity objects available for selection at the example electronic device 600. The opportunity summary region 602 presents a total number of available opportunity objects for selection. The opportunity summary region 602 presents a total number of available opportunity objects that have not yet been executed. The opportunity summary region 602 is configured to present only opportunity objects satisfying validation by the validation engine 430 or the integration engine 440. The opportunity summary region is configured to present only opportunity objects satisfying an availability determination by the opportunity availability controller 436.

The score summary region 604 is configured to present at least one metric associated with one or more opportunity objects available for election at the example electronic device 600. The score summary region 604 presents at least one execution value associated with the point module 524. The score summary region 604 presents an aggregation of a plurality of execution values associated with a corresponding plurality of point modules each associated with a particular point value. The opportunity list region 610 is configured to present at least one opportunity object 510 as a selectable GUI element. The opportunity list region includes one or more opportunity objects 612. The opportunity objects 612 each correspond to a distinct opportunity object 510 of a plurality available at the opportunity database 342.

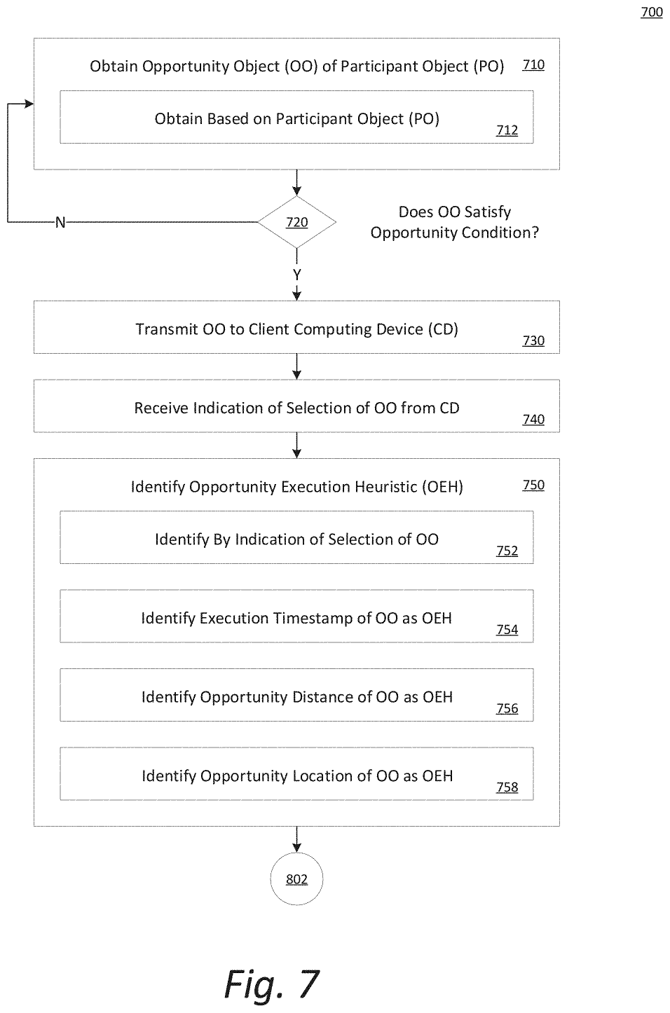

FIG. 7 illustrates an example method of generating a metric-based digital feed performance model, in accordance with present implementations. The method 700 can be performed by one or more component, system, device or module depicted in FIGS. 1A-6, including for example, a DPS or device 600. The method 700 begins at step 710.

At step 710, the DPS obtains at least one opportunity object associated with at least one participant object. The opportunity engine 320 obtains the opportunity object. In some implementations, step 710 includes step 712. At step 712, the DPS obtains the opportunity object based at least partially on the participant object. The opportunity engine 320 obtains the opportunity object associated with, linked to, or link, for example, a particular participant object. The method 700 then continues to step 720.

At step 720, the DPS determines whether the opportunity object satisfies an opportunity condition. The opportunity availability controller 436 determines whether the opportunity object satisfies an opportunity condition. In accordance with a determination that the opportunity object satisfies an opportunity condition, the method 700 continues to step 730. Alternatively, in accordance with a determination that the opportunity object does not satisfy an opportunity condition, the method 700 continues to step 710.

At step 730, the DPS transmits the opportunity object to at least one client computing device. In some implementations, at least one of the gateway engine 310 and the participant gateway 316 transmits the opportunity object to at least one of the participant computing devices 232. The method 700 then continues to step 740.

At step 740, the DPS receives at least one indication of a selection of an opportunity object from a client computing device. In some implementations, at least one of the gateway engine 310 and the participant gateway 316 receives at least one indication of a selection of an opportunity object from at least one of the participant computing devices 232. The DPS receives the indication from a client computing device to which the opportunity object is transmitted. The method 700 then continues to step 750.

At step 750, the DPS identifies at least one opportunity execution heuristic. In some implementations, at least one of the opportunity heuristic engine 334 and the validation engine 430 identifies the opportunity execution heuristic. In some implementations, step 750 includes at least one of steps 752, 754, 756 and 758. In some implementations, at least one of the validation engine 430 and the integration engine 440 aggregates and performs an aggregated validation by one or more of the validators 432, 434, 436 and 438 of the validation engine 430. At step 752, the DPS identifies the opportunity execution heuristic by the indication of the selection of the opportunity object. In some implementations, at least one of the opportunity heuristic engine 334 and the validation engine 430 identifies the opportunity execution heuristic by the indication. At step 754, the DPS identifies an execution timestamp associated with the opportunity object as the opportunity execution heuristic. In some implementations, at least one of the validation engine 430 and the timestamp validator 434 identifies the execution timestamp. At step 756, the DPS identifies an opportunity distance associated with the opportunity object as the opportunity execution heuristic. In some implementations, at least one of the validation engine 430 and the location validator 432 identifies the opportunity distance. At step 758, the DPS identifies an opportunity location associated with the opportunity object as the opportunity execution heuristic. In some implementations, In some implementations, at least one of the validation engine 430, the location validator 432, and the service validator 438 identifies the opportunity location. The method 700 then continues to step 802.

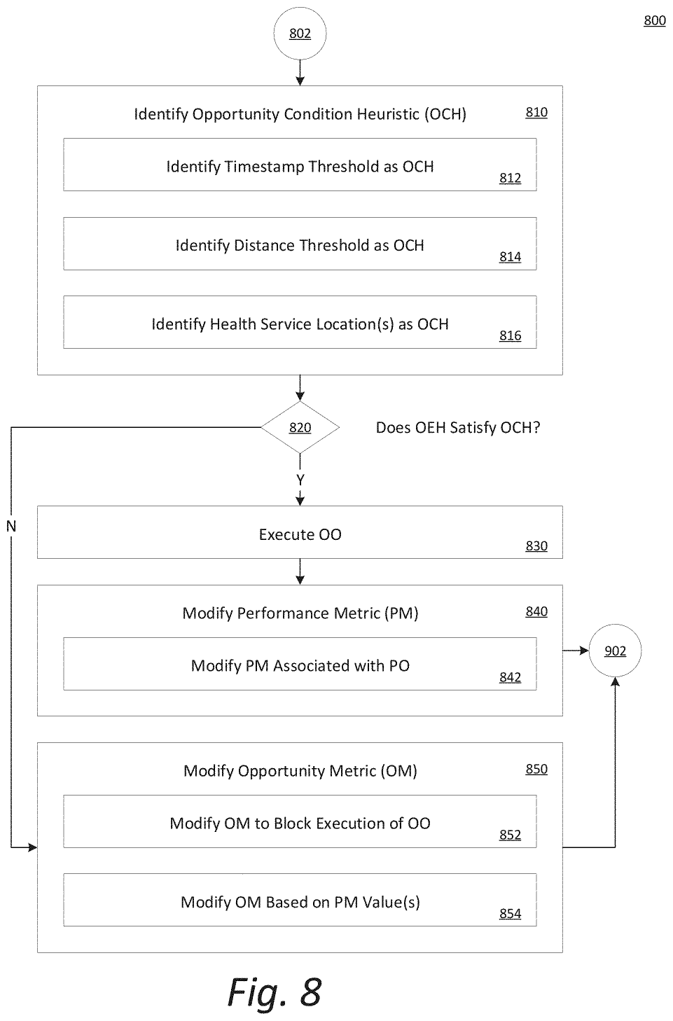

FIG. 8 illustrates an example method of generating a metric-based digital feed performance model further to the method of FIG. 7, in accordance with present implementations. The method 800 can be performed by one or more component, system, device or module depicted in FIGS. 1A-6, including for example, a DPS or device 600. The method 800 begins at step 802. The method 800 then continues to step 810.

At step 810, the DPS identifies at least one opportunity condition heuristic. In some implementations, at least one of the opportunity heuristic engine 334 and the validation engine 430 identifies the opportunity condition heuristic. In some implementations, step 810 includes at least one of steps 812, 814 and 816. At step 812, the DPS identifies a timestamp threshold as the opportunity condition heuristic. In some implementations, at least one of the validation engine 430 and the timestamp validator 434 identifies the timestamp threshold. At step 814, the DPS identifies a distance threshold as the opportunity condition heuristic. In some implementations, at least one of the validation engine 430 and the location validator 432 identifies the distance threshold. At step 816, the DPS identifies at least one health service location as the opportunity condition heuristic. In some implementations, at least one of the validation engine 430, the location validator 432, and the service validator 438 identifies the health service location. As one example, the DPS can evaluate the heuristic by the health service location by matching, or comparing, or link, for example, a location associated with a participant with one or more exact locations, addresses, geolocations, or geofences, or link, for example. The method 800 then continues to step 820.

At step 820, the DPS determines whether the opportunity execution heuristic satisfies the opportunity condition heuristic. In some implementations, at least one of the validation engine 430 and the integration engine 440 determines whether the opportunity execution heuristic satisfies the opportunity condition heuristic. In accordance with a determination that the opportunity execution heuristic satisfies the opportunity condition heuristic, the method 800 continues to step 830. Alternatively, in accordance with a determination that the opportunity execution heuristic does not satisfy the opportunity condition heuristic, the method 800 continues to step 850.

At step 830, the DPS executes the opportunity object. The opportunity engine executes the opportunity object. The method 800 then continues to step 840.

At step 840, the DPS modifies a performance metric. In some implementations, at least one of the opportunity metric engine 336, the participant metric controller 432, and the opportunity metric controller 434 modifies the performance metric. In some implementations, step 840 includes step 842. At step 842, the DPS modifies a performance metric associated with the participant object. The participant metric controller 432 modifies the performance metric associated with the participant object. The method 800 then continues to step 850.

At step 850, the DPS modifies an opportunity metric. The opportunity metric controller 434 modifies the opportunity metric. In some implementations, step 850 includes at least one of steps 852 and 854. At step 852, the DPS modifies the opportunity metric to block execution of the opportunity object. As one example, the opportunity metric controller 434 can modify an opportunity metric to reduce a score associated with the opportunity object or an opportunity object sharing one or more characteristics, or associations, or link, for example, with the opportunity object. At step 854, the DPS modifies the opportunity metric based at least partially on a value of the performance metric. The value of the performance metric is an absolute, relative or like score, or link, for example. The method 800 then continues to step 902.

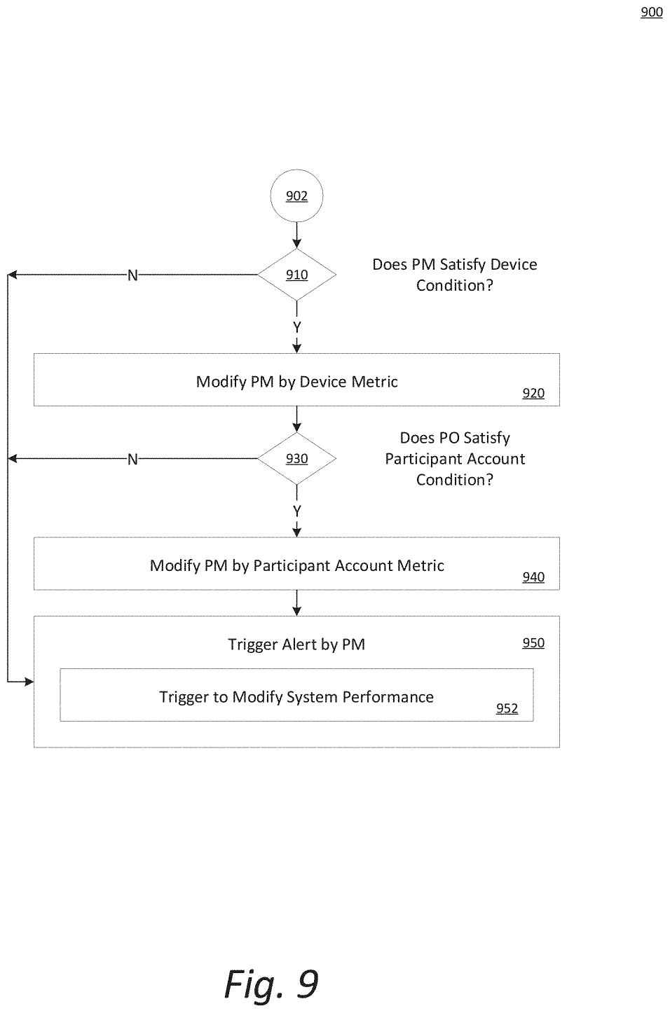

FIG. 9 illustrates an example method of generating a metric-based digital feed performance model further to the method of FIG. 8, in accordance with present implementations. The method 900 can be performed by one or more component, system, device or module depicted in FIGS. 1A-6, including for example, a DPS or device 600. The method 900 begins at step 902. The method 900 then continues to step 910.

At step 910, the DPS determines whether the performance metric satisfies a device condition. In some implementations, at least one of the device state engine 414, the device linker 416, the participant validator 436, and the service validator 438 determines whether the performance metric satisfies a device condition. In accordance with a determination that the performance metric satisfies a device condition, the method 900 continues to step 920. Alternatively, in accordance with a determination that the performance metric does not satisfy a device condition, the method 900 continues to step 950.

At step 920, the DPS modifies the performance metric by the device metric. In some implementations, at least one of the opportunity metric engine 336, the participant metric controller 432, and the opportunity metric controller 436 modifies the performance metric by the device metric. The method 900 then continues to step 930.

At step 930, the DPS determines whether the participant object satisfies a participant account condition. In some implementations, at least one of the validation engine 430 and the participant validator 436 determines whether the participant object satisfies a participant account condition. In accordance with a determination that the participant object satisfies a participant account condition, the method 900 continues to step 940. Alternatively, in accordance with a determination that the participant object does not satisfy a participant account condition, the method 900 continues to step 950.

At step 940, the DPS modifies the performance metric by the participant account metric. In some implementations, at least one of the opportunity metric engine 336 and the participant metric controller 432 modifies the performance metric by the participant account metric. The method 900 then continues to step 950.

At step 950, the DPS triggers an alert based at least partially on the performance metric. In some implementations, at least one of the opportunity engine 320, the scoring engine 330, and the opportunity heuristic engine 334 triggers an alert. In some implementations, step 950 includes step 952. At step 952, the DPS triggers the alert to modify system performance. The DPS modifies system performance by increasing available network or device bandwidth by reducing the number of opportunity objects being made available, or transmitted, or link, for example. The DPS reduces the number of opportunity objects being made available, or transmitted, or link, for example in accordance with one or more operations of the scoring engine 330, including but not limited to validation at the opportunity heuristic engine 334 and modification of availability by at least one of the opportunity metric engine 336 and the opportunity availability controller 436. The method 900 ends at step 950.

It should be understood that the systems described above can provide multiple ones of any or each of those components and these components can be provided on either a standalone machine or, in some implementations, on multiple machines in a distributed system. The systems and methods described above can be implemented as a method, apparatus or article of manufacture using programming or engineering techniques to produce software, firmware, hardware, or any combination thereof. In addition, the systems and methods described above can be provided as one or more computer-readable programs embodied on or in one or more articles of manufacture. The term “article of manufacture” as used herein is intended to encompass code or logic accessible from and embedded in one or more computer-readable devices, firmware, programmable logic, memory devices (e.g., EEPROMs, ROMs, PROMs, RAMs, SRAMs, etc.), hardware (e.g., integrated circuit chip, Field Programmable Gate Array (FPGA), Application Specific Integrated Circuit (ASIC), etc.), electronic devices, a computer readable non-volatile storage unit (e.g., CD-ROM, floppy disk, hard disk drive, etc.). The article of manufacture can be accessible from a file server providing access to the computer-readable programs via a network transmission line, wireless transmission media, signals propagating through space, radio waves, infrared signals, etc. The article of manufacture can be a flash memory card or a magnetic tape. The article of manufacture includes hardware logic as well as software or programmable code embedded in a computer readable medium that is executed by a processor. In general, the computer-readable programs can be implemented in any programming language, such as LISP, PERL, C, C++, C#, PROLOG, or in any byte code language such as JAVA. The software programs can be stored on or in one or more articles of manufacture as object code.

The herein described subject matter sometimes illustrates different components contained within, or connected with, different other components. It is to be understood that such depicted architectures are illustrative, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components and/or wirelessly interactable and/or wirelessly interacting components and/or logically interacting and/or logically interactable components

With respect to the use of plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

It will be further understood by those within the art that if a specific number of an introduced claim recitation is intended, such an intent will be explicitly recited in the claim, and in the absence of such recitation, no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general, such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Further, unless otherwise noted, the use of the words “approximate,” “about,” “around,” “substantially,” etc., mean plus or minus ten percent.

The foregoing description of illustrative implementations has been presented for purposes of illustration and of description. It is not intended to be exhaustive or limiting with respect to the precise form disclosed, and modifications and variations are possible in light of the above teachings or may be acquired from practice of the disclosed implementations. It is intended that the scope of the invention be defined by the claims appended hereto and their equivalents.

1. A system for generating a metric-based digital feed performance model, comprising:
a data processing system comprising memory and one or more processors to:
obtain by a real-time network interface compatible with a third-party administrator device, an opportunity object for a participant service identifier generated by an opportunity engine of the data processing system based on a participant object, the participant object associated with an individual participant user, the opportunity object including an action object associated with a third-party account at the third-party administrator device that is established for the individual participant user;
transmit, to a client computing device, the opportunity object responsive to the opportunity object satisfying an opportunity condition associated with the participant object;
receive, via an interface of the client computing device, an indication of a selection of the opportunity object;
identify, responsive to the indication of the selection, an opportunity execution heuristic associated with the participant object;
execute, responsive to a determination that the opportunity execution heuristic satisfies an opportunity condition heuristic, the opportunity object based on the action object; and
modify, responsive to the execution of the opportunity object, a performance metric associated with the participant object configured to trigger an alert to improve performance.

2. The system of claim 1, wherein the opportunity condition heuristic comprises a timestamp threshold, and the opportunity execution heuristic comprises an execution timestamp associated with the opportunity object.

3. The system of claim 2, wherein the data processing system is further operable to:
determine that the execution timestamp is before or at the timestamp threshold.

4. The system of claim 1, wherein the opportunity condition heuristic comprises a distance threshold, and the opportunity execution heuristic comprises at least one of an opportunity distance between a participant location associated with the participant object and an opportunity location associated with the opportunity object.

5. The system of claim 4, wherein the data processing system is further operable to:
determine that the opportunity distance is less than or equal to the distance threshold.

6. The system of claim 4, wherein the opportunity location comprises a location associated with a health service.

7. The system of claim 1, wherein the opportunity condition heuristic comprises an aggregation of a timestamp threshold and a distance threshold.

8. The system of claim 1, wherein the data processing system is further operable to:
modify, responsive to a determination that the opportunity execution heuristic does not satisfy the opportunity condition heuristic, an opportunity metric to block execution of the opportunity object.

9. The system of claim 8, wherein the data processing system is further operable to:
modify the opportunity metric based on a value of the performance metric.

10. The system of claim 1, wherein the data processing system is further operable to:
modify, responsive to a determination that the participant object satisfies a device condition, the performance metric according to a device metric.

11. The system of claim 1, wherein the data processing system is further operable to:
modify, responsive to a determination that the participant object satisfies a participant account condition, the performance metric according to a participant account metric associated with a participant account.

12. A method for generating a metric-based digital feed performance model, comprising:
obtaining by a real-time network interface compatible with a third-party administrator device, an opportunity object for a participant service identifier generated by an opportunity engine of the data processing system based on a participant object, the participant object associated with an individual participant user, the opportunity object including an action object associated with a third-party account at the third-party administrator device that is established for the individual participant user;
transmitting, to a client computing device, the opportunity object responsive to the opportunity object satisfying an opportunity condition associated with the participant object;
receiving, via an interface of the client computing device, an indication of a selection of the opportunity object;
identifying, responsive to the indication of the selection, an opportunity execution heuristic associated with the participant object;
executing, responsive to a determination that the opportunity execution heuristic satisfies an opportunity condition heuristic, the opportunity object based on the action object; and
modifying, responsive to the execution of the opportunity object, a performance metric associated with the participant object configured to trigger an alert to improve performance.

13. The method of claim 12, wherein the opportunity condition heuristic comprises a timestamp threshold, and the opportunity execution heuristic comprises an execution timestamp associated with the opportunity object.

14. The method of claim 13, further comprising:
determining that the execution timestamp is before or at the timestamp threshold.

15. The method of claim 12, wherein the opportunity condition heuristic comprises a distance threshold, and the opportunity execution heuristic comprises an opportunity distance between a participant location associated with the participant object and an opportunity location associated with the opportunity object.

16. The method of claim 15, further comprising:
determining that the opportunity distance is less than or equal to the distance threshold.

17. The method of claim 12, wherein the opportunity condition heuristic comprises an aggregation of a timestamp threshold and a distance threshold.

18. The method of claim 12, further comprising:
modifying, responsive to a determination that the opportunity execution heuristic does not satisfy the opportunity condition heuristic, an opportunity metric to block execution of the opportunity object.

19. A computer readable medium including one or more instructions stored thereon and executable by a processor to:
obtain by a real-time network interface compatible with a third-party administrator device, an opportunity object for a participant service identifier generated by an opportunity engine of the data processing system based on a participant object, the participant object associated with an individual participant user, the opportunity object including an action object associated with a third-party account at the third-party administrator device that is established for the individual participant user;
transmit, to a client computing device, the opportunity object responsive to the opportunity object satisfying an opportunity condition associated with the participant object;
receive, via an interface of the client computing device, an indication of a selection of the opportunity object;
identify, responsive to the indication of the selection, an opportunity execution heuristic associated with the participant object;
execute, responsive to a determination that the opportunity execution heuristic satisfies an opportunity condition heuristic, the opportunity object based on the action object; and
modify, responsive to the execution of the opportunity object, a performance metric associated with the participant object configured to trigger an alert to improve performance.

20. The computer readable medium of claim 19, wherein the computer readable medium further includes one or more instructions executable by a processor to:
modify, responsive to a determination that the opportunity execution heuristic does not satisfy the opportunity condition heuristic, an opportunity metric to block execution of the opportunity object.
